(19) United States
(12) Patent Application Publication
(10) Pub. No.: US 2026/0117328 A1
(43) Pub. Date: Apr. 30, 2026

(54) CHARACTERIZING CHIKUNGUNYA VIRUS

(71) Applicant: ILLUMINA, INC., San Diego, CA (US)

(72) Inventor: Jeffrey Koble, San Diego, CA (US)

(21) Appl. No.: 19/425,580

(22) Filed: Dec. 18, 2025

Related U.S. Application Data
(63) Continuation of application No. PCT/US2024/044460, filed on Aug. 29, 2024.
(60) Provisional application No. 63/536,333, filed on Sep. 1, 2023.

(30) Foreign Application Priority Data
Aug. 29, 2024 (WO) PCT/US2024/044460

(51) Int. Cl. C12Q 1/70; C12N 15/10; C12Q 1/6806; C12Q 1/6809; C12Q 1/6869

(52) U.S. Cl. CPC C12Q 1/701; C12N 15/1096; C12Q 1/6806; C12Q 1/6809; C12Q 1/6869; C12Q 2600/156

(57) ABSTRACT

This disclosure provides methods and compositions relating to the characterization of the chikungunya virus genome, including specific sets of oligonucleotide primers for the amplification of the chikungunya virus genome for downstream sequencing, alignment, and identification of variants in the chikungunya virus genome.

### CROSS-REFERENCE TO RELATED APPLICATION

This application is a bypass continuation of PCT/US2024/044460, which claims priority to and the benefit of U.S. Provisional Application No. 63/536,333, filed Sep. 1, 2023, the disclosures of which are incorporated herein by reference in their entireties.

### TECHNICAL FIELD

The present disclosure provides methods and compositions for amplification, sequencing, and characterization of the chikungunya virus genome.

### SEQUENCE LISTING

This application contains a Sequence Listing which has been submitted electronically as an XML file named 35629-0047WO1. The XML file, created on Aug. 26, 2024, is 158,456 bytes in size. The material in the XML file is hereby incorporated by reference in its entirety.

### BACKGROUND

Viral genomes can accumulate mutations during replication and propagation in a population. Viruses that have RNA as genetic material, e.g., SARS-COV-2, influenza, chikungunya, and dengue, accumulate mutations at a faster rate than viruses with DNA genomes. Accumulated mutations may confer varying degrees of pathogenicity or transmissibility and become more or less prevalent in a population. Genomic sequencing is useful for identifying variants of a virus in a specimen. Genomic surveillance, including sequencing, can be used by public health authorities to track the spread of viral variants and monitor changes in viral genomes in a population. This information can be used to better understand how circulating variants impact public health. The present disclosure provides methods and compositions that are particularly useful for characterizing the chikungunya virus genome.

### SUMMARY

The present disclosure relates to methods and compositions for characterizing the chikungunya virus genome. In particular, the methods and compositions of the disclosure relate to, for example, oligonucleotide primers for amplification of the chikungunya virus genome, sequencing methods, alignment of sequencing reads to a chikungunya virus reference genome, and detection and characterization of genomic variants in samples, e.g., biological samples, that may contain chikungunya virus genome samples. The disclosure provides methods of characterizing a chikungunya virus genome, including obtaining a nucleic acid of viral origin; obtaining virus-specific oligonucleotide primers (oligonucleotide sequences provided in Table 1 below); amplifying the nucleic acid using the oligonucleotide primers; sequencing the amplification products to produce sequencing reads; aligning the sequencing reads to a reference chikungunya genome to produce an alignment; and based on the alignment, identifying variants in the chikungunya virus genome.

In a first aspect, the disclosure provides methods of characterizing a chikungunya virus genome, the methods including: obtaining a sample including a nucleic acid of viral origin; obtaining a first plurality of oligonucleotide primers including sequences selected from the group consisting of SEQ ID NOs 2, 4, 5, 8, 9, 12, 13, 16, 17, 20, 21, 24, 25, 28, 29, 32, 33, 36, 37, 40, 41, 44, 45, 48, 49, 52, 53, 56, 57, 60, 61, 64, 65, 68, 69, 72, 73, and 76; obtaining a second plurality of oligonucleotide primers including sequences selected from the group consisting of SEQ ID NOs 3, 6, 7, 10, 11, 14, 15, 18, 19, 22, 23, 26, 27, 30, 31, 34, 35, 38, 39, 42, 43, 46, 47, 50, 51, 54, 55, 58, 59, 62, 63, 66, 67, 70, 71, 74, 75, and 77; amplifying the nucleic acid using the first and the second pluralities of oligonucleotide primers to produce first and second amplification products; sequencing the first and the second amplification products to produce sequencing reads; aligning the sequencing reads to a reference chikungunya genome to produce an alignment; and, based on the alignment, identifying one or more variants in the chikungunya virus genome compared to the reference chikungunya virus genome.

In some embodiments, the nucleic acid is RNA. In some embodiments, the methods further include, before the amplification step, reverse transcribing the RNA to produce cDNA. In some embodiments, the first plurality of oligonucleotide primers are configured for use as pairs, wherein the pairs are selected from the group consisting of SEQ ID NOs: 2 and 4, 5 and 8, 9 and 12, 13 and 16, 17 and 20, 21 and 24, 25 and 28, 29 and 32, 33 and 36, 37 and 40, 41 and 44, 45 and 48, 49 and 52, 53 and 56, 57 and 60, 61 and 64, 65 and 68, 69 and 72, and 73 and 76; and the second plurality of oligonucleotide primers are configured for use as pairs, wherein the pairs are selected from the group consisting of SEQ ID NOs: 3 and 6, 7 and 10, 11 and 14, 15 and 18, 19 and 22, 23 and 26, 27 and 30, 31 and 34, 35 and 38, 39 and 42, 43 and 46, 47 and 50, 51 and 54, 55 and 58, 59 and 62, 63 and 66, 67 and 70, 71 and 74, and 75 and 77.

In some embodiments, the reference chikungunya genome is about 80%, 85%, 90%, 95%, 96%, 87%, 98%, 99%, or 100% identical to SEQ ID NO: 1. In some embodiments, the sequencing is next generation sequencing (NGS). In some embodiments, the nucleic acid is obtained from a biological sample. In some embodiments, the sample includes blood or serum. In some embodiments, the methods further include, after the alignment step, quantifying a genomic coverage of the sequencing reads. In some embodiments, the genomic coverage is at least 50%, 55%, 60%, 65%, 70%, 75%, 80%, 85%, 95%, 96%, 97%, 98%, 99%, or 100% of the reference chikungunya genome at a minimum read depth of at least ten reads.

In some embodiments, the RNA is present in the sample in an amount equivalent to 200 copies of the chikungunya virus genome. In some embodiments, the genomic coverage is at least 50% of the reference chikungunya genome at a minimum read depth of at least ten reads. In some embodiments, the RNA is present in the sample in an amount equivalent to 500 copies of the chikungunya virus genome. In some embodiments, the genomic coverage is at least 75% of the reference chikungunya genome at a minimum read depth of at least ten reads. In some embodiments, the RNA is present in the sample in an amount equivalent to 800 copies of the chikungunya virus genome. In some embodiments, the genomic coverage is at least 80% of the reference chikungunya genome at a minimum read depth of at least ten reads. In some embodiments, the RNA is present in the sample in an amount equivalent to 5000 copies of the chikungunya virus genome. In some embodiments, the genomic coverage is at least 95% of the reference chikungunya genome at a minimum read depth of at least ten reads. In some embodiments, the methods further include distinguishing the chikungunya virus genome from a second nucleic acid of viral origin based on the identified variants.

In another aspect, the disclosure provides methods of detecting chikungunya virus RNA in a sample, e.g., for diagnostic, research, or surveillance purposes. The methods include: obtaining a sample; isolating RNA from the sample; obtaining a first plurality of oligonucleotide primers including sequences selected from the group consisting of SEQ ID NOs 2, 4, 5, 8, 9, 12, 13, 16, 17, 20, 21, 24, 25, 28, 29, 32, 33, 36, 37, 40, 41, 44, 45, 48, 49, 52, 53, 56, 57, 60, 61, 64, 65, 68, 69, 72, 73, and 76; obtaining a second plurality of oligonucleotide primers including sequences selected from the group consisting of SEQ ID NOs 3, 6, 7, 10, 11, 14, 15, 18, 19, 22, 23, 26, 27, 30, 31, 34, 35, 38, 39, 42, 43, 46, 47, 50, 51, 54, 55, 58, 59, 62, 63, 66, 67, 70, 71, 74, 75, and 77; reverse transcribing the RNA to produce cDNA amplifying the cDNA using the first and the second pluralities of oligonucleotide primers to produce first and second amplification products; sequencing the first and the second amplification products to produce sequencing reads; quantifying the sequencing reads; and, determining, based on the quantity of sequencing reads, a presence or absence of chikungunya virus RNA in the sample.

In some embodiments, the sample is a biological sample. In some embodiments, the biological sample includes blood or serum. In some embodiments, the sample is an environmental sample. In some embodiments, the environmental sample includes an extract from one or more mosquitos, or a wastewater or air filter sample.

In some embodiments, the first plurality of oligonucleotide primers are configured for use as pairs, wherein the pairs are selected from the group consisting of SEQ ID NOS: 2 and 4, 5 and 8, 9 and 12, 13 and 16, 17 and 20, 21 and 24, 25 and 28, 29 and 32, 33 and 36, 37 and 40, 41 and 44, 45 and 48, 49 and 52, 53 and 56, 57 and 60, 61 and 64, 65 and 68, 69 and 72, and 73 and 76; and the second plurality of oligonucleotide primers are configured for use as pairs, wherein the pairs are selected from the group consisting of SEQ ID NOs: 3 and 6, 7 and 10, 11 and 14, 15 and 18, 19 and 22, 23 and 26, 27 and 30, 31 and 34, 35 and 38, 39 and 42, 43 and 46, 47 and 50, 51 and 54, 55 and 58, 59 and 62, 63 and 66, 67 and 70, 71 and 74, and 75 and 77.

In some embodiments, the sequencing is next generation sequencing (NGS). In some embodiments, the methods further include assembling the sequencing reads to produce a consensus sequence. In some embodiments, the consensus sequence is produced if at least 35 amplicons are detected in the sequencing reads.

In another aspect, the disclosure provides kits including: one or more buffers; a reverse transcriptase; a first plurality of oligonucleotide primers including sequences selected from the group consisting of SEQ ID NOs 2, 4, 5, 8, 9, 12, 13, 16, 17, 20, 21, 24, 25, 28, 29, 32, 33, 36, 37, 40, 41, 44, 45, 48, 49, 52, 53, 56, 57, 60, 61, 64, 65, 68, 69, 72, 73, and 76; a second plurality of oligonucleotide primers including sequences selected from the group consisting of SEQ ID NOs 3, 6, 7, 10, 11, 14, 15, 18, 19, 22, 23, 26, 27, 30, 31, 34, 35, 38, 39, 42, 43, 46, 47, 50, 51, 54, 55, 58, 59, 62, 63, 66, 67, 70, 71, 74, 75, and 77; a DNA polymerase; and one or more library preparation agents.

The methods and compositions disclosed herein provide for successful amplification of the chikungunya virus genome for downstream sequencing resulting in high genomic coverage. Compared to previously available methods and compositions, the oligonucleotide primers provided by the disclosure result in increased sequencing coverage of the chikungunya virus genome. Further, the sets of oligonucleotide primers provided in Table 1 can provide an amplicon sequencing library that identifies variants in the chikungunya virus genome and is useful for distinguishing between strains of the chikungunya virus genome.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A is an Integrative Genomics Viewer window, table, and plot of genomic sequencing coverage for three technical replicates after amplification of a chikungunya virus genome using the oligonucleotide primers disclosed herein, with an RNA input equivalent to 200 copies of the chikungunya virus genome. Sequencing reads were aligned to the chikungunya virus reference genome NCBI Reference Sequence NC_004162.2 (SEQ ID NO: 1).

FIG. 1B is an Integrative Genomics Viewer window, table, and plot of genomic sequencing coverage for three technical replicates after amplification of a chikungunya virus genome using the oligonucleotide primers disclosed herein, with an RNA input equivalent to 500 copies of the chikungunya virus genome. Sequencing reads were aligned to the chikungunya virus reference genome NCBI Reference Sequence NC_004162.2 (SEQ ID NO: 1).

FIG. 1C is an Integrative Genomics Viewer window, table, and plot of genomic sequencing coverage for two technical replicates after amplification of a chikungunya virus genome using previously available oligonucleotide primers, with an RNA input equivalent to 200 copies of the chikungunya virus genome. Sequencing reads were aligned to the chikungunya virus reference genome NCBI Reference Sequence NC_004162.2 (SEQ ID NO: 1).

FIG. 1D is table and plot of genomic sequencing coverage for three technical replicates after amplification of a chikungunya virus genome using previously available oligonucleotide primers, with an RNA input equivalent to 500 copies of the chikungunya virus genome. Sequencing reads were aligned to the chikungunya virus reference genome NCBI Reference Sequence NC_004162.2 (SEQ ID NO: 1).

FIG. 1E is a table of genomic sequencing coverage after amplification of a chikungunya virus genome using the oligonucleotide primers disclosed herein, with an RNA input equivalent to 500 copies of the chikungunya virus genome. Sequencing reads were aligned to the chikungunya virus genome GenBank: MF580946.1 (99.966% coverage) and NCBI Reference Sequence NC_004162.2 (SEQ ID NO: 1) (98.452% coverage). A plot of genomic sequencing coverage for alignment to NCBI Reference Sequence NC_004162.2 is shown.

FIG. 2A is a plot of genomic sequencing coverage after amplification of a chikungunya virus genome using the oligonucleotide primers disclosed herein, with an RNA input equivalent to 500 copies of the chikungunya virus genome. Sequencing reads were aligned to the chikungunya virus reference genome NCBI Reference Sequence NC_004162.2 (SEQ ID NO: 1).

FIG. 2B is a plot of genomic sequencing coverage after amplification of a chikungunya virus genome using the oligonucleotide primers disclosed herein, with an RNA input equivalent to 800 copies of the chikungunya virus genome. Sequencing reads were aligned to the chikungunya virus reference genome NCBI Reference Sequence NC_004162.2 (SEQ ID NO: 1).

FIG. 2C is a plot of genomic sequencing coverage after amplification of a chikungunya virus genome using the oligonucleotide primers disclosed herein, with an RNA input equivalent to 5000 copies of the chikungunya virus genome. Sequencing reads were aligned to the chikungunya virus reference genome NCBI Reference Sequence NC_004162.2 (SEQ ID NO: 1).

### DETAILED DESCRIPTION

The disclosure provides amplicon-based library preparation methods for the sequencing and characterization of the chikungunya virus genome. The disclosure provides a set of oligonucleotide primers for the amplification of the chikungunya virus genome. Sequences of the oligonucleotide primers are provided in Table 1 below. The oligonucleotide primers are designed such that they are divided into two pools with alternate target genome regions, so that neighboring amplicons do not overlap within the same pool. Neighboring amplicons within each primer pool have a gap between each amplicon. The recommended analysis solution for the workflow is the DRAGEN™ Targeted Microbial Application (DTMA)™ implemented in BaseSpace™ for alignment, variant calling, and consensus genome output. Compared to previously available methods and compositions, the oligonucleotide primers provided by the disclosure result in increased sequencing coverage of the chikungunya virus genome.

The disclosure describes in further detail below the chikungunya virus; methods of virus surveillance; oligonucleotide primers, PCR amplification, and sequencing; workflow of the methods disclosed herein; and kits for performing the methods disclosed herein.

### Chikungunya Virus

Chikungunya virus has an approximately 12 kb positive sense RNA genome that encodes four non-structural proteins (nsP1-4), with five structural proteins (C, E3, E2, 6K, and E1) expressed from subgenomic RNA synthesized in infected cells. The genome has a short 5′ untranslated region and a longer 3′ untranslated region comprising stem-loop structures and direct repeats that are thought to be associated with adaptation of the virus to mosquito hosts. The genome is packed into virions that are similar to those of other alphaviruses. The cellular receptors for chikungunya virus remain unknown. Chikungunya virions are internalized by clathrin-mediated endocytosis, but the available evidence also suggests that the entry pathway might be cell-type specific or that multiple pathways are used. The epidemiology, molecular virology, virus-host interactions, immunological responses, animal models, and potential antiviral therapies and vaccines for chikungunya virus are reviewed in, for example, Burt F J, et al. Chikungunya virus: an update on the biology and pathogenesis of this emerging pathogen. Lancet Infect Dis. 2017 April; 17(4):e107-e117.

The complete single-stranded RNA reference genome for chikungunya virus is publicly available and accessible at the website of the National Center for Biotechnology Information (NCBI), listed as NCBI Reference Sequence NC_004162.2 and provided as SEQ ID NO: 1 below:

(SEQ ID NO: 1)
atggctgcgtgagacacacgtagcctaccagtttcttactgctctactctgcaaagcaagagattaagaac
ccatcatggatcctgtgtacgtggacatagacgctgacagcgcctttttgaaggccctgcaacgtgcgtac
cccatgtttgaggtggaacctaggcaggtcacaccgaatgaccatgctaatgctagagcgttctcgcatct
agctataaaactaatagagcaggaaattgatcccgactcaaccatcctggatattggtagtgcgccagcaa
ggaggatgatgtcggacaggaagtaccactgcgtttgcccgatgcgcagtgcagaagatcccgagagactc
gccaattatgcgagaaagctagcatctgccgcaggaaaagtcctggacagaaacatctctggaaagatcgg
ggacttacaagcagtaatggccgtgccagacacggagacgccaacattctgcttacacacagatgtatcat
gtagacagagagcagacgtcgcgatataccaagacgtctatgctgtacacgcacccacgtcgctataccac
caggcgattaaaggggtccgattggcgtactgggtagggtttgacacaaccccgttcatgtacaatgccat
ggcgggtgcctacccctcatactcgacaaattgggcagatgagcaggtactgaaggctaagaacataggat
tatgttcaacagacctgacggaaggtagacgaggcaaattgtctattatgagaggaaaaaagctagaaccg
tgcgaccgtgtgctgttctcagtagggtcaacgctctacccggaaagccgtaagctacttaagagctggca
cctaccatcggtgttccatttaaagggcaagctcagcttcacatgccgctgtgatacagtggtttcgtgcg
aaggctacgtcgttaagagaataacgatgagcccaggcctttacggaaaaaccacagggtatgcggtaacc
caccacgcagacggattcctgatgtgcaagaccaccgacacggttgacggcgaaagagtgtcattctcggt
gtgcacgtacgtgccggcgaccatttgtgatcaaatgaccggcatccttgctacagaagtcacgccggagg
atgcacagaagctgttggtggggctgaaccagagaatagtggttaacggcagaacgcaacggaatacgaac
accatgaaaaactatatgattcccgtggtcgcccaagccttcagtaagtgggcaaaggagtgccggaaaga
catggaagatgaaaaactcctgggggtcagagaaagaacactgacctgctgctgtctatgggcatttaaga
agcagaaaacacacacggtctacaagaggcctgatacccagtcaattcagaaggttcaggccgagtttgac
agctttgtggtaccgagcctgtggtcgtccgggttgtcaatcccgttgaggactagaatcaaatggttgtt
aagcaaggtgccaaaaaccgacctgaccccatacagcggggacgcccaagaagcccgggacgcagaaaaag
aagcagaggaagaacgagaagcagaactgactcttgaagccctaccaccccttcaggcagcacaggaagat
gttcaggtcgaaatcgacgtggaacagcttgaggacagagcgggtgcaggaataatagagactccgagagg
agctatcaaagttactgcccaaccaacagaccacgtcgtgggagagtacttggttctttccccgcagaccg
tactacgtagccaaaagcttagcctgattcacgctttggcggagcaagtgaagacgtgcacgcacagcgga
cgagcagggaggtatgcggtcgaagcgtacgacggcagagtcctagtgccctcaggctacgcaatctcgcc
tgaagacttccagagcctaagcgaaagcgcaacgatggtgtacaacgaaagagagttcgtaaacagaaagc
tacaccatattgcgatgcatggaccagccctgaacaccgacgaagagtcgtatgagctggtgagggcagag
aggacagaacacgagtacgtctacgacgtggaccagagaagatgctgtaagaaggaagaagctgcaggact
ggtactggtgggcgacttgactaatccgccctaccacgaattcgcatatgaagggctaaaaatccgccctg
cctgcccatacaaaattgcagtcataggagtcttcggagtaccaggatctggcaagtcagctattatcaag
aacctagttaccaggcaagacctggtgactagcggaaagaaagaaaactgccaagaaatcaccaccgacgt
gatgagacagagaggtctagagatatctgcacgtacggttgactcgctgctcttgaatggatgtaacagac
cagtcgacgtgttgtacgtagacgaggcgtttgcgtgccactctggaacgttacttgcattgatcgccttg
gtgagaccaagacagaaagttgtactttgtggtgacccgaagcagtgcggcttcttcaatatgatgcagat
gaaagtcaactataatcacaacatctgcacccaagtgtaccacaaaagtatctccaggcggtgtacactgc
ctgtgactgccattgtgtcatcgttgcattacgaaggcaaaatgcgcactacgaatgagtacaacaagccg
attgtagtggacactacaggctcaacaaaacctgaccctggagatctcgtgttaacgtgcttcagaggatg
ggttaaacaactgcaaattgactatcgtggacacgaggtcatgacagcagccgcatcccaagggttaacca
gaaaaggagtttacgcagttaggcaaaaagttaacgaaaacccgctttatgcatcaacgtcagagcacgtc
aacgtactcctaacgcgtacggaaggtaaactggtatggaagacactctccggtgacccgtggataaagac
gctgcagaacccaccgaaaggaaacttcaaagcaactattaaggagtgggaggtggagcatgcatcaataa
tggcgggcatctgcagtcaccaaatgacctttgatacattccaaaacaaagccaacgtttgttgggctaag
agtttggtccctatcctcgaaacagcggggataaaactaaacgacaggcagtggtcccagataattcaagc
cttcaaagaagacaaagcatattcacccgaagtagccctgaatgaaatatgcacgcgcatgtatggggtgg
atctagacagcgggctattttctaaaccgttggtgtctgtgtattacgcggataaccactgggataatagg
cctggagggaagatgttcggattcaaccccgaggcagcatccattctagaaagaaagtatccatttacaaa
agggaagtggaacatcaacaagcagatctgcgtgactaccaggaggatagaagacttcaaccctaccacca
acattataccggccaacaggagactaccacactcattagtggccgaacaccgcccagtaaaaggggaaaga
atggaatggctggttaacaagataaacggccaccacgtgctcctggtcagtggctgtagccttgcactgcc
tactaagagagtcacttgggtagcgccactaggtgtccgcggagcggactatacatacaacctagagttgg
gtctgccagcaacgcttggtaggtatgacctagtggtcataaacatccacacaccttttcgcatacaccat
tatcaacagtgcgtagaccacgcaatgaaactgcaaatgctcgggggtgactcattgagactgctcaaacc
gggggctctctattgatcagagcatatggttacgcagatagaaccagtgaacgagtcatctgcgtattgg
gacgcaagtttagatcatctagagcgttgaaaccaccatgtgtcaccagcaacactgagatgttttttcta
ttcagcaactttgacaatggcagaaggaatttcacaactcatgtcatgaacaatcaactgaatgcagcctt
tgtaggacaggccacccgagcaggatgtgcaccgtcgtaccgggtaaaacgcatggatatcgcgaagaacg
atgaagagtgcgtagtcaacgccgccaaccctcgcgggttaccaggtgacggtgtttgcaaggcagtatac
aaaaaatggccggagtcctttaagaacagtgcaacaccagtgggaaccgcaaaaacagtcatgtgcggtac
gtatccagtaatccacgccgttggaccaaacttctctaattattcggagtctgaaggggaccgagaattgg
cggctgcctatcgagaagtcgcaaaggaggtaactagactgggagtaaatagtgtagctatacctctcctc
tccacaggtgtatactcaggagggaaagacaggctgacccagtcactgaaccacctctttacagccatgga
ctcgacggatgcagacgtggtcatctactgccgcgacaaagaatgggagaagaaaatatctgaggccatac
agatgcggacccaagtggagctgctggatgagcacatctccatagactgcgatgttgttcgcgtgcaccct
gacagcagcttggcaggcagaaaaggatacagcaccacggaaggcgcactgtactcatatctagaagggac
ccgttttcaccaaacggcagtggatatggcagagatatatactatgtggccaaagcaaacagaggccaacg
agcaagtttgcctatatgccctgggggaaagtattgaatcgatcaggcagaaatgcccggtggatgatgca
gatgcatcatctcccccgaaaactgtcccgtgcctctgccgttacgccatgacaccagaacgcgttacccg
acttcgcatgaaccatgtcacaagcataattgtgtgttcttcgtttccccttccaaagtacaaaatagaag
gagtgcaaaaagtcaaatgctccaaggtaatgctatttgaccacaacgtgccatcgcgcgtaagtccaagg
gaatacagaccttcccaggagtctgtacaggaagcgagtacgaccacgtcactgacgcatagccaattcga
tctaagcgttgacggcaagatactgcccgtcccgtcagacctggatgctgacgccccagccctagaaccag
cccttgacgacggggcgatacacacgttgccatctgcaaccggaaaccttgcggccgtgtctgactgggta
atgagcaccgtacctgtcgcgccgcccagaagaaggcgagggagaaacctgactgtgacatgcgacgagag
agaagggaatataacacccatggctagcgtccgattctttagggcagagctgtgtccagtcgtacaagaaa
cagcggagacgcgtgacacagctatgtctcttcaggcaccgccgagtaccgccacggaactgagtcacccg
ccgatctccttcggtgcaccaagcgagacgttccccatcacatttggggacttcaacgaaggagaaatcga
aagcttgtcttctgagctactaactttcggagacttcctacccggagaagtggatgatttgacagatagcg
actggtccacgtgctcagacacggacgacgagttacgactagacagggcaggtgggtatatattctcgtcg
gacactggtccaggtcatttacaacagaagtcagtacgccagtcagtgctgccggtgaacaccctggagga
agtccacgaggagaagtgttacccacctaagctggatgaagcaaaggagcaactactacttaagaaactcc
aggagagtgcatccatggccaacagaagcaggtatcagtcgcgcaaagtagaaaacatgaaagcaacaatc
atccagagactaaagagaggctgtagattatacttaatgtcagagaccccaaaagtccctacctaccggac
cacatatccggcgcctgtgtactcgcctccgattaacgtccgactgtccaaccccgagtccgcagtggcag
catgcaatgagttcttggctagaaactatccaactgtttcatcataccaaatcaccgacgagtatgatgca
tatctagacatggtggacgggtcggagagttgtctggaccgagcgacattcaatccgtcaaaacttaggag
ctacccaaaacagcacgcttaccacgcgccctccatcagaagcgctgtaccgtccccattccagaacacac
tacagaatgtactggcagcagccacgaaaagaaactgcaacgtcacacagatgagggaattacccactttg
gactcagcagtattcaacgtggagtgtttcaaaaaattcgcatgcaaccaagaatactgggaagaatttgc
tgccagccctatcaggataacaactgagaatttaacaacctatgttactaaactaaaggggccaaaagcag
cagcgctatttgcaaaaacccataatctgctgccactgcaggaagtgccaatggataggttcacagtagac
atgaaaagggatgtgaaggtgactcctggtacaaagcacacagaggaaagacctaaggtacaggttataca
ggcggctgaacccttggcaacagcatacctatgtgggattcacagagagctggttaggaggctgaacgccg
tcctcctacccaatgtacatacactatttgacatgtctgccgaggatttcgatgccatcatagccgcacac
tttaagccaggagacactgttttagaaacggacatagcctcctttgataagagccaagatgattcacttgc
gcttactgctttaatgctgttagaggatttaggggtggatcactccctgttggacttgatagaggctgctt
tcggagagatttccagctgtcatctaccgacaggtacgcgcttcaagttcggcgccatgatgaaatctggt
atgttcctaactctgttcgtcaacacactgctaaatatcaccatcgccagccgagtgctggaagatcgtct
gacaaaatccgcgtgcgcagccttcatcggcgacgacaacataatacatggagtcgtctccgatgaattga
tggcagccagatgcgccacttggatgaacatggaagtgaagatcatagatgcagttgtatcccagaaagcc
ccttacttttgtggagggtttatactgcacgatatcgtgacaggaacagcttgcagagtggcagacccgct
aaaaaggctatttaaactgggcaaaccgctagcggcaggtgacgaacaagatgaggatagaagacgagcgc
tggctgacgaagtggtcagatggcaacgaacagggctaattgatgagttggagaaagcggtatactctagg
tatgaagtgcagggtatatcagttgtggtaatgtccatggccacctttgcaagctccagatccaacttcga
gaagctcagaggacccgtcgtaactttgtacggcggtcctaaataggtacgcactacagctacctattttg
cagaagccgacagtaagtacctaaacactaatcagctacaatggagttcatcccaacccaaactttttaca
acaggaggtaccagcctcgaccctggactccgcgccctactatccaagtcatcaggcccagaccgcgcccg
cagaggcaagctgggcaacttgcccagctgatctcagcagttaataaactgacaatgcgcgcggtacccca
acagaagccacgcaagaatcggaagaataagaagcaaaagcaaaagcagcaggcgccacaaaacaacacaa
accaaaagaagcagccacctaaaaagaaaccagctcaaaagaaaaagaagccgggccgcagagagaggatg
tgcatgaaaatcgaaaatgactgtattttcgaagtcaagcacgaaggtaaggtaacaggttacgcgtgctt
ggtgggggacaaagtaatgaaaccagcacacgtaaaggggaccatcgataacgcggacctggccaaattgg
cctttaagcggtcatctaagtacgaccttgaatgcgcgcagatacccgtgcacatgaagtccgacgcttcg
aagttcacccatgagaaaccggaggggtactacaactggcaccacggagcagtacagtactcaggaggccg
gttcaccatccctacaggtgcgggcaaaccaggggacagcggtagaccgatcttcgacaacaagggacgcg
tggtggccatagtcttaggaggagctaatgaaggagcccgtacagccctctcagtggtgacctggaataaa
gacattgtcactaaaatcacccctgagggagccgaagagtggagtcttgccatcccagttatgtgcctgtt
ggcaaataccacgttcccctgctcccagcccccttgcataccctgctgctacgaaaaggaaccggaggaaa
ccctacgcatgcttgaggacaacgtcatgagacctgggtactatcagctgctacaagcatcattaacatgt
tctccccaccgccagcgacgcagcaccaaggacaacttcaatgtctataaagccacaagaccatacctagc
tcactgtcccgactgtggagaagggcactcgtgccatagtcccgtagcactagaacgcatcagaaatgaag
cgacagacgggacgctgaaaatccaggtctccttgcaaattggaatagggacggatgatagccatgattgg
accaagctgcgttacatggacaatcacataccagcagacgcagggagggccgggctatttgtaagaacatc
agcaccatgcacgattactggaacaatgggacacttcatcctggcccgatgtccgaaaggagaaactctga
cggtgggattcactgacagtaggaagattagtcactcatgtacgcacccatttcaccacgaccctcctgtg
ataggccgggaaaaattccattcccgaccgcagcacggtaaagagctaccttgcagcacgtacgtgcagag
caacgccgcaactgccgaggagatagaggtacacatgcccccagacacccctgatcgcacattgctgtcac
aacagtccggcaacgtaaagatcacagtcaatagtcagacggtgcggtataagtgtaattgcggtggctca
aatgaaggactaataactacagataaagtgattaataactgcaaggttgatcaatgtcatgccgcggtcac
caatcacaaaaagtggcagtataactcccctctggtcccgcgtaacgctgaactcggggaccgaaaaggaa
aaattcacatcccgtttccgctggcaaatgtaacatgcatggtgcctaaagcaaggaaccccaccgtgacg
tacgggaaaaaccaagtcatcatgctactgtatcctgaccacccaacactcctgtcctaccggagtatggg
agaagaaccaaactatcaagaagagtgggtgacgcacaagaaggaggtcgtgctaaccgtgccgactgaag
ggctcgaggttacgtggggcaacaacgagccgtataagtattggccgcagttatctgcaaacggtacagcc
cacggccacccgcatgagataatcttgtactattatgagctgtaccctactatgactgtagtagttgtgtc
agtggcctcgttcatactcctgtcgatggtgggtatggcagtggggatgtgcatgtgtgcacgacgcagat
gcatcacaccatacgaactgacaccaggagctaccgtccctttcctgcttagcctaatatgctgcatcaga
acagctaaagcggccacataccaagaggctgcggtatacctgtggaacgagcagcaacctttgttttggct
acaagcccttattccgctggcagccctgattgtcctatgcaactgtctgagactcttaccatgctgttgta
aaacgttggcttttttagccgtaatgagcatcggtgcccacactgtgagcgcgtacgaacacgtaacagtg
atcccgaacacggtgggagtaccgtataagactctagtcaacagaccgggctacagccccatggtactgga
gatggagctactgtcagtcactttggagccaacgctatcgcttgattacatcacgtgcgaatacaaaaccg
tcatcccgtctccgtacgtgaaatgctgcggtacagcagagtgcaaggacaaaaacctacctgactacagc
tgtaaggtcttcaccggcgtctacccatttatgtggggcggcgcctactgcttctgcgacgctgaaaacac
gcaattgagcgaagcacatgtggagaagtccgaatcatgcaaaacagaatttgcatcagcatacagggctc
ataccgcatccgcatcagctaagctccgcgtcctttaccaaggaaataacatcactgtaactgcctatgca
aacggcgaccatgccgtcacagttaaggacgccaaattcattgtggggccaatgtcttcagcctggacacc
ttttgacaacaaaatcgtggtgtacaaaggtgacgtttacaacatggactacccgccctttggcgcaggaa
gaccaggacaatttggcgatatccaaagtcgcacgcctgagagcaaagacgtctatgctaacacacaactg
gtactgcagagaccggctgcgggtacggtacacgtgccatactctcaggcaccatctggctttaagtattg
gttaaaagaacgaggggcgtcgctacagcacacagcaccatttggctgccaaatagcaacaaacccggtaa
gagcgatgaactgcgccgtagggaacatgcccatctccatcgacataccggatgcggccttcactagggtc
gtcgacgcgccctctttaacggacatgtcatgcgaggtaccagcctgcacccattcctcagactttggggg
cgtcgccattattaaatatgcagtcagcaagaaaggcaagtgtgcggtgcattcgatgaccaacgccgtca
ctatccgggaagctgagatagaagttgaagggaattctcagctgcaaatctctttctcgacggccttggcc
agcgccgaattccgcgtacaagtctgttctacacaagtacactgtgcagccgagtgccaccctccgaagga
ccacatagtcaactacccggcgtcacataccaccctcggggtccaggacatttccgctacggcgatgtcat
gggtgcagaagatcacgggaggtgtgggactggttgtcgctgttgcagcactgattctaatcgtggtgcta
tgcgtgtcgttcagcaggcactaacttgacgactaagcatgaaggtatatgtgtcccctaagagacacacc
gtatatagctaataatctgtagatcaaagggctatataacccctgaatagtaacaaaatacaaaatcacta
aaaattataaaaaaaaaaaaaaaaaaacagaaaaatatataaataggtatacgtgtcccctaagagacaca
ttgtatgtaggtgataagtatagatcaaagggccgaacaacccctgaatagtaacaaaatataaaaattaa
taaaaatcataaaatagaaaaaccataaacagaagtagttcaaagggctataaaaacccctgaatagtaac
aaaacataaaactaataaaaatcaaatgaataccataattggcaaacggaagagatgtaggtacttaagct
tcctaaaagcagccgaactcactttgagatgtaggcatagcataccgaactcttccacgattctccgaacc
cacagggacgtaggagatgttattttgtttttaatatttc

### Virus Detection and Surveillance

Monitoring viruses, both newly emerging viruses and well-established viruses, is an important measure taken to control the spread and impact of viruses in animal and human populations. In some embodiments, the compositions and methods disclosed herein can be used for rapid analysis of many viral samples to identify which isolate or isolates of chikungunya virus are present in an individual subject and to identify sequence variation between isolates. Early sequence monitoring of many isolates in parallel can be used to rapidly identify isolates and mutations. Some isolates of a given virus, e.g., chikungunya virus, may have more severe phenotypes than other isolates, for example, higher morbidity or mortality rates and/or greater drug resistance. When a new outbreak occurs, rapid re-sequencing of isolates from affected individuals can be used to identify individuals infected with isolates known to have more dangerous phenotypes and steps can be taken to aggressively contain the spread of those isolates. For example, a plurality of individuals may be identified as having symptoms of a disease, e.g., chikungunya infection.

Samples that may contain virus particles can be isolated from each of the affected individuals and re-sequenced to identify variation between samples. The variation may be compared against a reference sequence, e.g., the chikungunya reference sequence provided herein as SEQ ID NO: 1. In some embodiments, the sequencing data produced by the compositions and methods disclosed herein are compared to a chikungunya reference sequence that is about 80%, 85%, 90%, 95%, 96%, 87%, 98%, 99%, or 100% identical to SEQ ID NO: 1. In some embodiments, identified variants in the sequencing data are compared against a database of variation and phenotypes associated with these variations to identify individuals who have strains of the virus that are known to be, for example, more easily transmitted than other strains. Aggressive steps may be taken to insure that those individuals infected with the more transmittable strain are isolated so that transmission is limited. Resources may also be allocated to identifying subjects, e.g., people or animals that were likely to have been contacted by individuals with the easily transmitted strain to minimize the spread of strains with more severe phenotypes.

Additional methods of virus surveillance utilizing the amplification and sequencing methods disclosed herein include isolating chikungunya virus RNA from patient sputum, lung lavage, nasal swabs, air filter samples, wastewater samples, blood bank samples, and blood samples isolated from mosquito populations.

Chikungunya virus RNA sequencing results generated by the amplification and sequencing methods disclosed herein can be used to report virus sequences in samples for public health and research applications. The amplification and sequencing methods disclosed herein can also be used to perform chikungunya virus strain typing for monitoring virus evolution and epidemiology in populations of, e.g., humans and mosquitos.

### Oligonucleotide Primers, PCR Amplification, and Sequencing

The disclosure provides an amplicon-based library preparation for the amplification, sequencing, and characterization of the chikungunya virus genome. The genome of the chikungunya virus is amplified using a virus-specific oligonucleotide primer set provided herein. The resulting amplicon library can be sequenced, for example, using next-generation sequencing (NGS). In some embodiments, the resulting NGS data are analyzed using the DRAGEN Targeted Microbial Application (DTMA) analysis pipeline implemented in BaseSpace (Illumina, San Diego, CA) for alignment, variant calling, and consensus genome output.

The chikungunya virus genomic sample can be amplified by a variety of mechanisms, some of which employ the polymerase chain reaction (PCR). See, for example, PCR Technology. Principles and Applications for DNA Amplification (Ed. H. A. Erlich, Freeman Press, NY, N. Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683.202, 4,683,195, 4,800,159 4,965,188, and 5,333,675, each of which is incorporated herein by reference in their entireties for all purposes.

The methods disclosed herein also employ conventional biology methods, software, and systems. Computer software products that are part of the present disclosure typically include computer readable medium having computer-executable instructions for performing the logic steps of the methods disclosed herein. The computer executable instructions may be written in a suitable computer language or combination of several languages. Basic computational biology methods that may be used in the methods disclosed herein are described in, for example Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics. Application in Biological Science and Medicine (CRC Press, London, 2000) and Ouelette and BZevanis Bioinformatics: A Practical Guide for Analysis of Gene and Proteins (Wiley & Sons, Inc., 2″ ed., 2001).

Assays for the amplification of chikungunya virus genomic samples can be designed by any number of computational primer design tools known in the art. For example, primers for PCR amplification of the chikungunya virus genome can be designed to tile across the entirety of the genome in overlapping segments.

In some embodiments, as input, a primer design tool can use a FASTA file containing one or more reference genomes, e.g., the chikungunya virus genome provided herein as SEQ ID NO: 1. In some embodiments, a chikungunya genome sequence that is about 80%, 85%, 90%, 95%, 96%, 87%, 98%, 99%, or 100% identical to SEQ ID NO: 1 is used as an input for primer design. A desired PCR amplicon length can be specified. A PCR amplicon length can be, for example, 200, 300, 400, 500, 600, 700, 800, 900, 1000, 1100, 1200, 1300, 1400, 1500, 1600, 1700, 1800, 1900, or 2,000 nucleotides. A desired length of overlap between neighboring amplicons can be specified. A desired length of overlap between neighboring amplicons can be, for example, 50, 55, 60, 65, 70, 75, 80, 85, 90, 95, or 100 nucleotides. In some embodiments, oligonucleotide primers are designed such that they are divided into two pools with alternate target genome regions, so that neighboring amplicons do not overlap within the same pool. Neighboring amplicons within each primer pool have a gap between each amplicon.

For RNA viruses a first reverse transcriptase step can be used to generate double stranded DNA from the single stranded RNA. The amplicon-based library preparation disclosed herein for the chikungunya virus can be designed to re-sequence the approximately 12,000 base sequence published for the chikungunya virus, provided herein as SEQ ID NO: 1. In some embodiments, the amplicon-based library preparation can be designed to re-sequence an entire genome, such as the genome of the chikungunya virus; one or more regions of a genome, for example, selected regions of a genome such as those coding for a protein or RNA of interest; a conserved region from multiple genomes, or multiple genomes, such as the genome of a first chikungunya virus isolate and the genome of a second chikungunya virus isolate, or the genome of chikungunya virus and the genome of a different virus.

The synthesis of oligonucleotide primers is well known and routine in the art. The primers may be routinely made through the well-known technique of, for example, solid phase synthesis. Equipment for such synthesis is sold by several vendors including, for example, Applied BioSystems (Foster City, Calif.). Any other means for such synthesis known in the art can additionally or alternatively be employed.

In some embodiments, the primers used for amplification hybridize to and amplify genomic DNA, DNA of bacterial plasmids, DNA of DNA viruses or DNA reverse transcribed from RNA of an RNA virus. Methods of amplifying RNA to produce cDNA using reverse transcriptase are well known to those with ordinary skill in the field. In some embodiments, various computer software programs may be used to aid in the design of primers for amplification reactions such as Primer Premier 5 (Premier Biosoft, Palo Alto, Calif.); OLIGO Primer Analysis Software (Molecular Biology Insights, Cascade, Colo.); Primer3 (Untergasser A, et al. Primer3--new capabilities and interfaces. Nucleic Acids Res. 2012 August; 40(15):e115.); and Primalscheme (Quick J, et al. Multiplex PCR method for MinION and Illumina sequencing of Zika and other virus genomes directly from clinical samples. Nat Protoc. 2017 June; 12(6):1261-1276.)

These programs allow the user to input desired hybridization conditions such as melting temperature of a primer-template duplex for example. In some embodiments, an in silico PCR search algorithm, such as (ePCR) is used to analyze primer specificity across a plurality of template sequences which can be readily obtained from public sequence databases such as GenBank for example. An existing RNA structure search algorithm (Macke et al., Nucl. Acids Res., 2001, 29, 4724-4735, which is incorporated herein by reference in its entirety) has been modified to include PCR parameters such as hybridization conditions, mismatches, and thermodynamic calculations (SantaLucia, Proc. Natl. Acad. Sci. U.S.A., 1998, 95, 1460-1465, which is incorporated herein by reference in its entirety). This algorithm also provides information on primer specificity of the selected primer pairs. In some embodiments, the hybridization conditions applied to the algorithm can limit the results of primer specificity obtained from the algorithm.

In some embodiments, the melting temperature threshold for the primer template duplex is specified to be 35° C., 40° C., 45° C., 50° C., 55° C., 60° C., 65° C., 70° C., or a higher temperature. In some embodiments, the melting temperature for the primer template duplex is specified to be about 60-70° C. In some embodiments the number of acceptable mismatches is specified to be 10, 9, 8, 7, 6, 5, 4, 3, 2, 1, or zero mismatches. In some embodiments, the buffer components and concentrations and primer concentrations may be specified and incorporated into the algorithm, for example, an appropriate primer concentration is about 250 nM and appropriate buffer components are 50 mM sodium or potassium and 1.5 mM Mg.

One with ordinary skill in the art of design of amplification primers will recognize that a given primer need not hybridize to the target nucleic acid with 100% complementarity to effectively prime the synthesis of a complementary nucleic acid strand in an amplification reaction. Moreover, a primer may hybridize over one or more segments such that intervening or adjacent segments are not involved in the hybridization event (e.g., a loop structure or a hairpin structure). The oligonucleotide primers disclosed herein can comprise at least 70%, at least 75%, at least 80%, at least 85%, at least 90%, at least 95%, at least 99%, or 100% sequence identity with any of the primers listed in Table 1. Thus, in some embodiments, an extent of variation of 70% to 100%, or any range therewithin, of the sequence identity is possible relative to the specific primer sequences disclosed in Table 1. Determination of sequence identity is described in the following example: a primer 20 nucleobases in length which is identical to another 20 nucleobase primer but having two non-identical residues has 18 of 20 identical residues (18/20=0.9 or 90% sequence identity). In another example, a primer 15 nucleobases in length having all residues identical to a 15 nucleobase segment of primer 20 nucleobases in length would have 15/20=0.75 or 75% sequence identity with the 20 nucleobase primer.

Percent homology, sequence identity or complementarity, can be determined by, for example, the Gap program (Wisconsin Sequence Analysis Package, Version 8 for UNIX, Genetics Computer Group, University Research Park, Madison Wis.), using default settings, which uses the algorithm of Smith and Waterman (Adv. Appl. Math., 1981, 2, 482-489). In some embodiments, complementarity of primers with respect to the conserved priming regions of viral nucleic acid is between about 70% and about 75%. In other embodiments, homology, sequence identity or complementarity, is between about 75% and about 80%. In yet other embodiments, homology, sequence identity, or complementarity, is at least 85%, at least 90%, at least 92%, at least 94%, at least 95%, at least 96%, at least 97%, at least 98%, at least 99% or is 100%. In some embodiments, the primers described herein comprise at least 70%, at least 75%, at least 80%, at least 85%, at least 90%, at least 92%, at least 94%, at least 95%, at least 96%, at least 98%, or at least 99%, or 100% (or any range therewithin) sequence identity with the primer sequences specifically disclosed herein.

One with ordinary skill is able to calculate percent sequence identity or percent sequence homology and able to determine the effects of variation of primer sequence identity on the function of the primer in its role in priming synthesis of a complementary strand of nucleic acid for production of an amplification product of a corresponding segment of virus genome. In some embodiments, the oligonucleotide primers are 13 to 35 nucleobases in length (13 to 35 linked nucleotide residues). These embodiments comprise oligonucleotide primers 13, 14, 15, 16, 17, 18, 19, 20, 21, 22, 23, 24, 25, 26, 27, 28, 29, 30, 31, 32, 33, 34 or 35 nucleobases in length, or any range therewithin.

In some embodiments, the amplification steps of the methods disclosed herein are performed using one or more pairs of oligonucleotide primers provided in Table 1. The pairs of oligonucleotide primers can be selected from the group consisting of SEQ ID NOs: 2 and 4; SEQ ID NOs: 5 and 8; SEQ ID NOs: 9 and 12; SEQ ID NOs: 13 and 16; SEQ ID NOs: 17 and 20; SEQ ID NOs: 21 and 24; SEQ ID NOs: 25 and 28; SEQ ID NOs: 29 and 32; SEQ ID NOs: 33 and 36; SEQ ID NOs: 37 and 40; SEQ ID NOs: 41 and 44; SEQ ID NOs: 45 and 48; SEQ ID NOs: 49 and 52; SEQ ID NOs: 53 and 56; SEQ ID NOs: 57 and 60; SEQ ID NOs: 61 and 64; SEQ ID NOs: 65 and 68; SEQ ID NOs: 69 and 72; SEQ ID NOs: 73 and 76; SEQ ID NOs: 3 and 6; SEQ ID NOs: 7 and 10; SEQ ID NOs: 11 and 14; SEQ ID NOs: 15 and 18; SEQ ID NOs: 19 and 22; SEQ ID NOs: 23 and 26; SEQ ID NOs: 27 and 30; SEQ ID NOs: 31 and 34; SEQ ID NOs: 35 and 38; SEQ ID NOs: 39 and 42; SEQ ID NOs: 43 and 46; SEQ ID NOs: 47 and 50; SEQ ID NOs: 51 and 54; SEQ ID NOs: 55 and 58; SEQ ID NOs: 59 and 62; SEQ ID NOs: 63 and 66; SEQ ID NOs: 67 and 70; SEQ ID NOs: 71 and 74; and SEQ ID NOs: 75 and 77.

In some embodiments, oligonucleotide primers are designed such that they are divided into two pools with a first pool comprising oligonucleotide primer pairs SEQ ID NOs: 2 and 4; SEQ ID NOs: 5 and 8; SEQ ID NOs: 9 and 12; SEQ ID NOs: 13 and 16; SEQ ID NOs: 17 and 20; SEQ ID NOs: 21 and 24; SEQ ID NOs: 25 and 28; SEQ ID NOs: 29 and 32; SEQ ID NOs: 33 and 36; SEQ ID NOs: 37 and 40; SEQ ID NOs: 41 and 44; SEQ ID NOs: 45 and 48; SEQ ID NOs: 49 and 52; SEQ ID NOs: 53 and 56; SEQ ID NOs: 57 and 60; SEQ ID NOs: 61 and 64; SEQ ID NOs: 65 and 68; SEQ ID NOs: 69 and 72; and SEQ ID NOs: 73 and 76, and a second pool comprising oligonucleotide primer pairs SEQ ID NOs: 3 and 6; SEQ ID NOs: 7 and 10; SEQ ID NOs: 11 and 14; SEQ ID NOs: 15 and 18; SEQ ID NOs: 19 and 22; SEQ ID NOs: 23 and 26; SEQ ID NOs: 27 and 30; SEQ ID NOs: 31 and 34; SEQ ID NOs: 35 and 38; SEQ ID NOs: 39 and 42; SEQ ID NOs: 43 and 46; SEQ ID NOs: 47 and 50; SEQ ID NOs: 51 and 54; SEQ ID NOs: 55 and 58; SEQ ID NOs: 59 and 62; SEQ ID NOs: 63 and 66; SEQ ID NOs: 67 and 70; SEQ ID NOs: 71 and 74; and SEQ ID NOs: 75 and 77.

TABLE 1
Primers for Amplification of the Chikungunya Virus Genome

| | Primer | | |
|---|---|---|---|
| Oligonucleotide Name | pool | Sequence | SEQ ID NO |
| CHIK-ILM-PP_1_LEFT | 1 | AGACACACGTAGCCTACCAGTT | 2 |
| CHIK-ILM-PP_2_LEFT | 2 | TGCAGAAGATCCCGAGAGACTC | 3 |
| CHIK-ILM-PP_1_RIGHT | 1 | TCCAGAGATGTTTCTGTCCAGGA | 4 |
| CHIK-ILM-PP_3_LEFT | 1 | GCCTACCCCTCATACTCGACAA | 5 |
| CHIK-ILM-PP_2_RIGHT | 2 | TACCTTCCGTCAGGTCTGTTGA | 6 |
| CHIK-ILM-PP_4_LEFT | 2 | AGGCCTTTACGGAAAAACCACA | 7 |
| CHIK-ILM-PP_3_RIGHT | 1 | AATGACACTCTTTCGCCGTCAA | 8 |
| CHIK-ILM-PP_5_LEFT | 1 | AAAACTCCTGGGGGTCAGAGAA | 9 |
| CHIK-ILM-PP_4_RIGHT | 2 | AGACCGTGTGTGTTTTCTGCTT | 10 |
| CHIK-ILM-PP_6_LEFT | 2 | TTGAAGCCCTACCACCCCTT | 11 |
| CHIK-ILM-PP_5_RIGHT | 1 | TATTATTCCTGCACCCGCTCTG | 12 |
| CHIK-ILM-PP_7_LEFT | 1 | GAAGACTTCCAGAGCCTAAGCG | 13 |
| CHIK-ILM-PP_6_RIGHT | 2 | TCCATGCATCGCAATATGGTGT | 14 |
| CHIK-ILM-PP_8_LEFT | 2 | CAGGATCTGGCAAGTCAGCTAT | 15 |
| CHIK-ILM-PP_7_RIGHT | 1 | TGGCAGTTTTCTTTCTTTCCGC | 16 |
| CHIK-ILM-PP_9_LEFT | 1 | ACTATAATCACAACATCTGCACCCA | 17 |
| CHIK-ILM-PP_8_RIGHT | 2 | ATGACACAATGGCAGTCACAGG | 18 |
| CHIK-ILM-PP_10_LEFT | 2 | CCGCTTTATGCATCAACGTCAG | 19 |
| CHIK-ILM-PP_9_RIGHT | 1 | CCGGAGAGTGTCTTCCATACCA | 20 |
| CHIK-ILM-PP_11_LEFT | 1 | CAAGCCTTCAAAGAAGACAAAGCA | 21 |
| CHIK-ILM-PP_10_RIGHT | 2 | AATAGCCCGCTGTCTAGATCCA | 22 |
| CHIK-ILM-PP_12_LEFT | 2 | CACTCATTAGTGGCCGAACACC | 23 |
| CHIK-ILM-PP_11_RIGHT | 1 | CACGTGGTGGCCGTTTATCT | 24 |
| CHIK-ILM-PP_13_LEFT | 1 | GGGTGACTCATTGAGACTGCTC | 25 |
| CHIK-ILM-PP_12_RIGHT | 2 | GCAGATGACTCGTTCACTGGTT | 26 |
| CHIK-ILM-PP_14_LEFT | 2 | CGAAGAACGATGAAGAGTGCGT | 27 |
| CHIK-ILM-PP_13_RIGHT | 1 | GTTCTTAAAGGACTCCGGCCAT | 28 |
| CHIK-ILM-PP_15_LEFT | 1 | ACTGAACCACCTCTTTACAGCC | 29 |
| CHIK-ILM-PP_14_RIGHT | 2 | TCTTCTCCCATTCTTTGTCGCG | 30 |
| CHIK-ILM-PP_16_LEFT | 2 | CAAAGCAAACAGAGGCCAACG | 31 |
| CHIK-ILM-PP_15_RIGHT | 1 | TTTCGGGGGAGATGATGCATC | 32 |
| CHIK-ILM-PP_17_LEFT | 1 | CAGACCTTCCCAGGAGTCTGTA | 33 |
| CHIK-ILM-PP_16_RIGHT | 2 | CAGTATCTTGCCGTCAACGCTT | 34 |
| CHIK-ILM-PP_18_LEFT | 2 | CATGGCTAGCGTCCGATTCTTT | 35 |
| CHIK-ILM-PP_17_RIGHT | 1 | GGTGCCTGAAGAGACATAGCTG | 36 |
| CHIK-ILM-PP_19_LEFT | 1 | CTAGACAGGGCAGGTGGGTATA | 37 |
| CHIK-ILM-PP_18_RIGHT | 2 | AGCACTGACTGGCGTACTGA | 38 |
| CHIK-ILM-PP_20_LEFT | 2 | ATATCCGGCGCCTGTGTACT | 39 |
| CHIK-ILM-PP_19_RIGHT | 1 | GCCAAGAACTCATTGCATGCTG | 40 |
| CHIK-ILM-PP_21_LEFT | 1 | AACTGCAACGTCACACAGATGA | 41 |
| CHIK-ILM-PP_20_RIGHT | 2 | AGTATTCTTGGTTGCATGCGAAT | 42 |
| CHIK-ILM-PP_22_LEFT | 2 | TATACAGGCGGCTGAACCCTT | 43 |
| CHIK-ILM-PP_21_RIGHT | 1 | GTATGTACATTGGGTAGGAGGACG | 44 |
| CHIK-ILM-PP_23_LEFT | 1 | AAGTTCGGCGCCATGATGAAAT | 45 |
| CHIK-ILM-PP_22_RIGHT | 2 | CGGCTGGCGATGGTGATATTTA | 46 |
| CHIK-ILM-PP_24_LEFT | 2 | GCAGACCCGCTAAAAAGGCT | 47 |
| CHIK-ILM-PP_23_RIGHT | 1 | AGCCAGCGCTCGTCTTCTAT | 48 |
| CHIK-ILM-PP_25_LEFT | 1 | GCAGAAGCCGACAGTAAGTACC | 49 |
| CHIK-ILM-PP_24_RIGHT | 2 | AGGCTGGTACCTCCTGTTGTA | 50 |
| CHIK-ILM-PP_26_LEFT | 2 | ACCAGCTCAAAAGAAAAAGAAGCC | 51 |
| CHIK-ILM-PP_25_RIGHT | 1 | ACCTTCGTGCTTGACTTCGAAA | 52 |
| CHIK-ILM-PP_27_LEFT | 1 | CGGAGCAGTACAGTACTCAGGA | 53 |
| CHIK-ILM-PP_26_RIGHT | 2 | GCGTCCCTTGTTGTCGAAGATC | 54 |
| CHIK-ILM-PP_28_LEFT | 2 | AACGTCATGAGACCTGGGTACT | 55 |
| CHIK-ILM-PP_27_RIGHT | 1 | AAGTTGTCCTTGGTGCTGCG | 56 |
| CHIK-ILM-PP_29_LEFT | 1 | GGCCGGGCTATTTGTAAGAACA | 57 |
| CHIK-ILM-PP_28_RIGHT | 2 | CCGTCAGAGTTTCTCCTTTCGG | 58 |
| CHIK-ILM-PP_30_LEFT | 2 | GATCGCACATTGCTGTCACAAC | 59 |
| CHIK-ILM-PP_29_RIGHT | 1 | TGTAGTTATTAGTCCTTCATTTGAGCCA | 60 |
| CHIK-ILM-PP_31_LEFT | 1 | ACTGTATCCTGACCACCCAACA | 61 |
| CHIK-ILM-PP_30_RIGHT | 2 | GCGTCACCCACTCTTCTTGATA | 62 |
| CHIK-ILM-PP_32_LEFT | 2 | TATGGCAGTGGGGATGTGCAT | 63 |
| CHIK-ILM-PP_31_RIGHT | 1 | TTAGGCTAAGCAGGAAAGGGAC | 64 |
| CHIK-ILM-PP_33_LEFT | 1 | ACGTAACAGTGATCCCGAACAC | 65 |
| CHIK-ILM-PP_32_RIGHT | 2 | AGCTCCATCTCCAGTACCATGG | 66 |
| CHIK-ILM-PP_34_LEFT | 2 | GCACATGTGGAGAAGTCCGAAT | 67 |
| CHIK-ILM-PP_33_RIGHT | 1 | TTAGCTGATGCGGATGCGGTAT | 68 |
| CHIK-ILM-PP_35_LEFT | 1 | GCCTGAGAGCAAAGACGTCTAT | 69 |
| CHIK-ILM-PP_34_RIGHT | 2 | CCTGAGAGTATGGCACGTGTAC | 70 |
| CHIK-ILM-PP_36_LEFT | 2 | TGCACCCATTCCTCAGACTTTG | 71 |
| CHIK-ILM-PP_35_RIGHT | 1 | GATAGTGACGGCGTTGGTCATC | 72 |
| CHIK-ILM-PP_37_LEFT | 1 | TGTCATGGGTGCAGAAGATCAC | 73 |
| CHIK-ILM-PP_36_RIGHT | 2 | CGTCAAGTTAGTGCCTGCTGAA | 74 |
| CHIK-ILM-PP_38_LEFT | 2 | ACCCCTGAATAGTAACAAAATACAAAATCA | 75 |
| CHIK-ILM-PP_37_RIGHT | 1 | GCCCTTTGAACTACTTCTGTTTATGG | 76 |
| CHIK-ILM-PP_38_RIGHT | 2 | AACATCTCCTACGTCCCTGTGG | 77 |

### Workflow

The workflow of the amplicon-based library preparation methods disclosed herein can include or consist of the following procedures: chikungunya virus RNA extraction, cDNA synthesis, target amplification, library preparation, library pooling, sequencing, and analysis.

Chikungunya virus RNA can be extracted from a biological sample by any means known in the art. A biological sample can be, for example, blood or serum extracted from a patient. In some embodiments, chikungunya virus RNA is extracted from patient, blood, serum, sputum, lung lavage, nasal swabs. In some embodiments, chikungunya virus RNA is extracted from environmental sources including air filter samples and wastewater samples. In some embodiments, chikungunya virus RNA is extracted from sources of blood including blood banks, or is isolated from mosquito populations that may be carrying the virus. Commercially available methods of RNA extraction include, for example, Quick-DNA/RNA Viral MagBead Kit (Zymo Research, #R2141) or QIAamp Viral RNA Mini Kit (Qiagen, part #52906).

In some embodiments, the following steps of the workflow are performed with a chikungunya virus RNA input equivalent to 100 copies of the chikungunya virus genome, 200 copies, 300 copies, 400 copies, 500 copies, 600 copies, 700 copies, 800 copies, 900 copies, 1000 copies, 2000 copies, 3000 copies, 4000 copies, or 5000 or more copies.

DNA complementary to the chikungunya RNA (i.e., cDNA) can be reverse transcribed by reverse transcriptase with random hexamers. Next, the chikungunya virus genome present in the sample can be amplified using two separate PCR reactions that are then pooled together. In some embodiments, one PCR reaction utilizes oligonucleotide primers designated “Primer Pool 1” in Table 1, and the other PCR reaction utilizes oligonucleotide primers designated “Primer Pool 2” in Table 1. In some embodiments, the pooled amplified fragments undergo tagmentation to further fragment and tag amplicons with adapter sequences. Post-tagmentation yield can be normalized due to saturation of the bead-linked transposome by typical amplicon inputs. The adapter-tagged amplicons can undergo a second round of PCR amplification using a PCR master mix and unique index adapters. After amplification, indexed libraries can be pooled and cleaned using purification beads.

The pooled library product can be quantified using a fluorescent dye with concentration determined by comparison to a DNA standard curve. The pooled library product can be sequenced by any number of commercially available sequencing platforms. In some embodiments, pooled libraries are clustered onto a flow cell, and then sequenced using sequencing by synthesis (SBS) chemistry on, for example, the NovaSeq 6000 Sequencing System using the NovaSeq Xp S4 and SP flow cells, NextSeq 500 System, NextSeq 550 System, NextSeq 550Dx Instrument in RUO mode, or NextSeq 2000 System. The amplification and sequencing workflow disclosed herein can be scaled up or down to accommodate different numbers of samples. For examples, 1536 to 3072 results can be processed on the NovaSeq 6000 system in 12 hours using two SP or S4 reagent kits, or 384 results in 12 hours using the NextSeq 2000 or the NextSeq 500/550/550Dx (in RUO mode) HO reagent kit.

SBS chemistry uses a reversible-terminator method to detect single, fluorescently labeled deoxynucleotide triphosphate (dNTP) bases as they are incorporated into growing DNA strands. During each sequencing cycle, a single dNTP is added to the nucleic acid chain. The dNTP label serves as a terminator for polymerization. After each dNTP incorporation, the fluorescent dye is imaged to identify the base, and then cleaved to allow incorporation of the next nucleotide. Four reversible terminator-bound dNTPs (A, G, T, and C) are present as single, separate molecules. As a result, natural competition minimizes incorporation bias. During the primary analysis, base calls are made directly from signal intensity measurements during each sequencing cycle, resulting in base-by-base sequencing. A quality score is assigned to each base call.

In some embodiments, the Illumina® DRAGEN® Pipeline analyzes sequencing results to detect the presence of chikungunya virus RNA in each sample. As a quality control feature, an internal control consisting of, for example, one or more human mRNA targets can be included in every sample. Analysis can be performed locally using the Illumina DRAGEN or on BaseSpace® Sequence Hub. In some embodiments, the Illumina DRAGEN Pipeline performs small variant calling and generates a consensus sequence in FASTA format. Analysis can include a quantification of sequencing coverage depth. Sequencing coverage depth refers to the average number of sequencing reads that align to, or cover, each base in a sequenced sample. The Lander/Waterman equation is a method for calculating coverage (C) based on read length (L), number of reads (N), and haploid genome length (G): C=LN/G.

Analysis can include a quantification of genomic coverage. Genomic coverage refers to the breadth of coverage of a target genome, which is defined as the percentage of target bases that are sequenced a given number of times. For example, a genome sequencing study may sequence a genome to 30× average depth and achieve a 95% breadth of coverage of the reference genome at a minimum depth of ten reads. In some embodiments, the methods disclosed herein yield a genomic coverage of 80%, 85%, 95%, 96%, 97%, 98%, 99%, or 100% of the chikungunya virus genome at a minimum read depth of ten reads.

In some embodiments, when amplification is successful and sequencing reads are generated, a consensus sequence is generated from the sequencing reads. In some embodiments, a contig is assembled from the sequencing reads, wherein the sequencing reads overlap in a way that provides a contiguous representation of the chikungunya virus genome. In some embodiments, a consensus sequence is generated and reported when at least 30, 31, 32, 33, 34, 35, 36, 37, 38, 39, or 40 different amplicons are detected in the sequencing reads.

### Kits

The present disclosure also provides kits for carrying out the methods described herein. In some embodiments, the kit comprises a sufficient quantity of one or more primer pairs, e.g., one or more of the primer pairs provided in Table 1, to perform an amplification reaction on a DNA reverse transcribed from the chikungunya virus genome for downstream sequencing.

In some embodiments, the kit comprises a sufficient quantity of reverse transcriptase, a DNA polymerase, suitable nucleoside triphosphates (e.g., dNTPs), a DNA ligase, and/or reaction buffer, or any combination thereof, for the amplification processes described above. A kit can further include instructions pertinent for the particular embodiment of the kit, such as instructions describing the primer pairs and amplification conditions for operation of the methods described herein. A kit can also comprise amplification reaction containers such as microcentrifuge tubes and the like. A kit can also comprise reagents or other materials for isolating chikungunya virus RNA or identifying resulting amplicons from amplification, including, for example, detergents, solvents, and/or ion exchange resins, which may be linked to magnetic beads. In some embodiments, the kit includes a computer program stored on a computer formatted medium (such as a compact disk or portable USB disk drive, for example) comprising instructions that direct a processor to analyze data obtained from the use of the primer pairs disclosed herein. In some embodiments, the kits of the present disclosure contain all of the reagents sufficient to carry out one or more of the methods described herein.

In some embodiments, the kit further comprises one or more library preparation agents. Library preparation agents can include beads, buffers (e.g., elution buffers, reaction buffers, tagmentation buffers) and reaction mixes (e.g., mixes including enzymes, polymerases, transcriptases, reverse transcriptases).

In some embodiments, the kit comprises one or more of Illumina® Tune Beads and Stop Tagment Buffer 2 HT. In some embodiments, the kit further comprises one or more of enrichment bead-linked transposomes (BLT), elution buffer, resuspension buffer, and tagmentation wash buffer. In some embodiments, the kit further comprises one or more of elution prime fragment 3HC mix, enhanced PCR mix, first strand mix, Illumina PCR mix, a reverse transcriptase, and tagmentation buffer 1. In some embodiments, the kit further comprises a positive control RNA sample.

### EXAMPLES

The invention is further described in the following examples, which do not limit the scope of the invention described in the claims.

### Example 1: Amplification and Sequencing of the Chikungunya Virus Genome

Oligonucleotide primers listed in Table 1 were computationally designed using a software tool with NCBI Reference Sequence NC_004162.2 as the template genome for primer design. After we designed the primers, DNA oligonucleotides were synthesized according to the design, normalized to 100 μM, pooled into two primer pools as described in Table 1, and diluted to 10 μM for each pool to generate two overlapping sets of amplicons. RNA input equivalent to 200 and 500 copies of the chikungunya virus genome was used for two separate amplification and sequencing experiments. Three technical replicates were performed for each experiment. After amplification, amplicon libraries were denatured and diluted from a pooled library according to the Illumina® NextSeq® 500 and 550 Sequencing Systems Denature and Dilute Libraries Guide (15048776). Libraries were sequenced on the Illumina NextSeq® 550 instrument at 2×149 base pair read length, unless stated otherwise, and normalized to 1M paired-end read depth based on current sequencing recommendations. Analysis was executed using the DRAGEN™ Viral Lineage App v0.4.0.

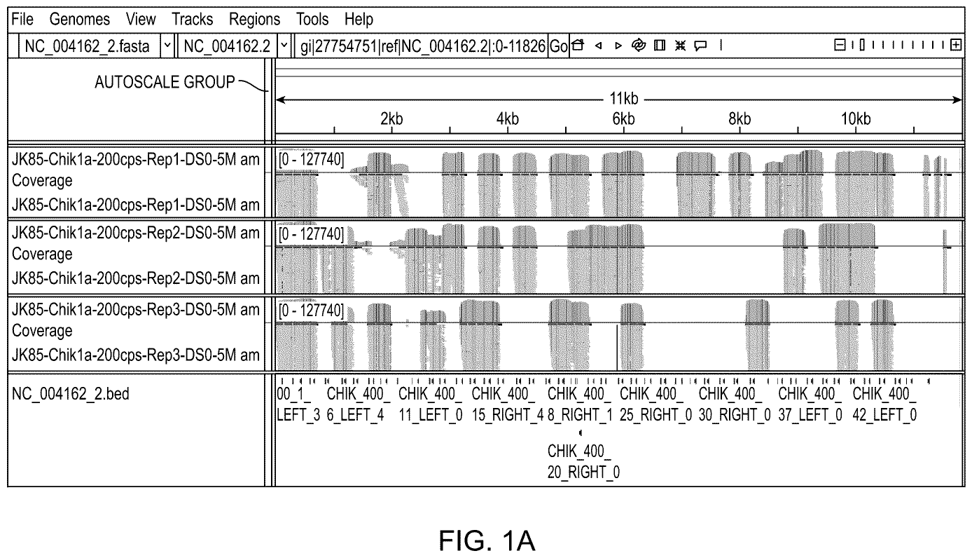

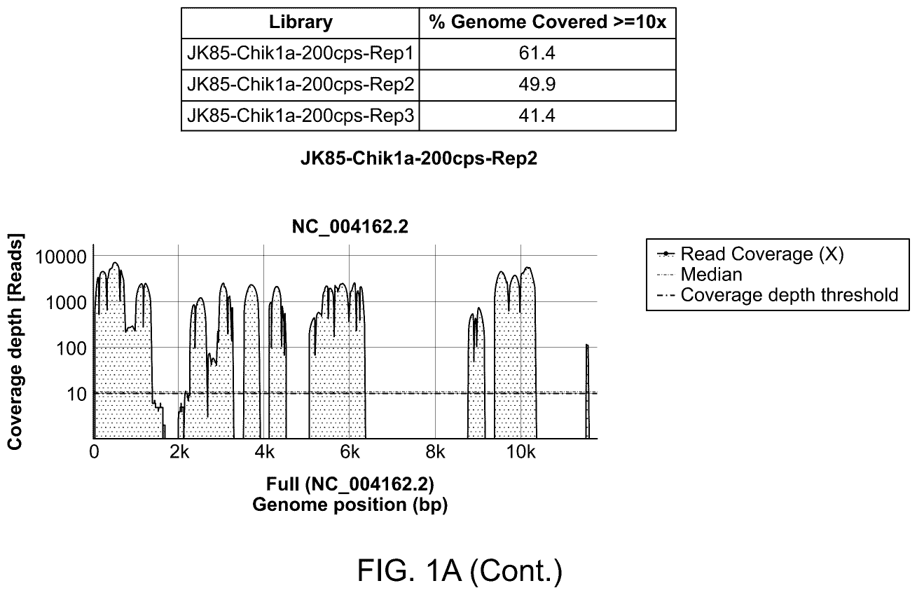

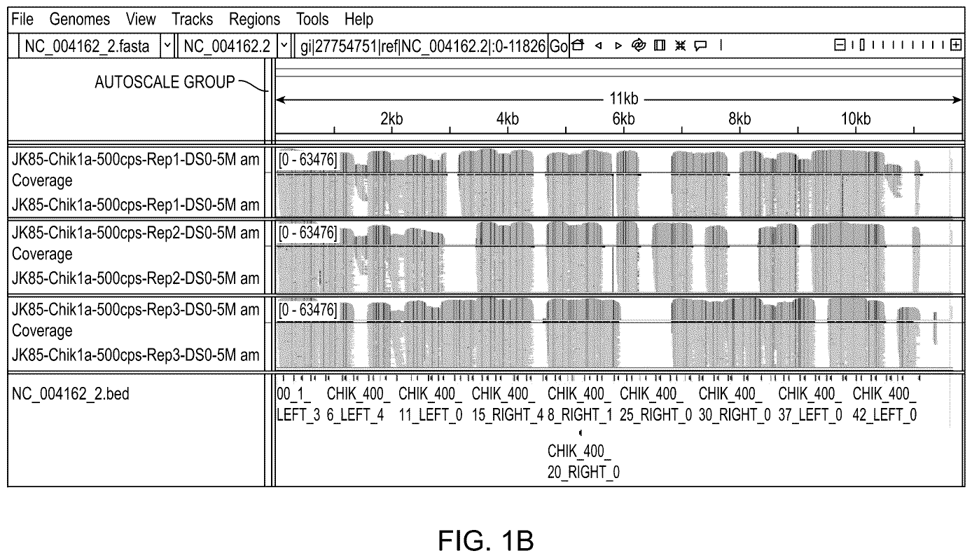

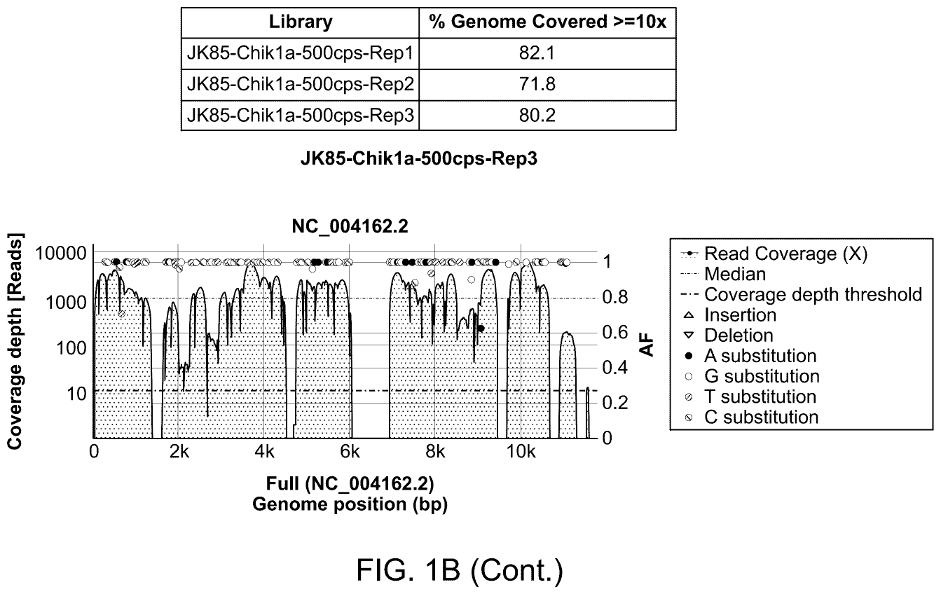

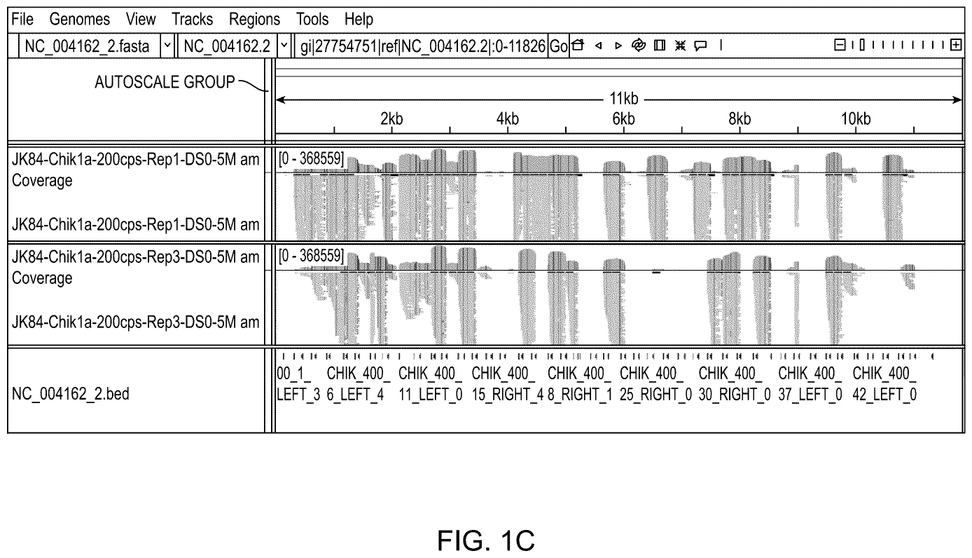

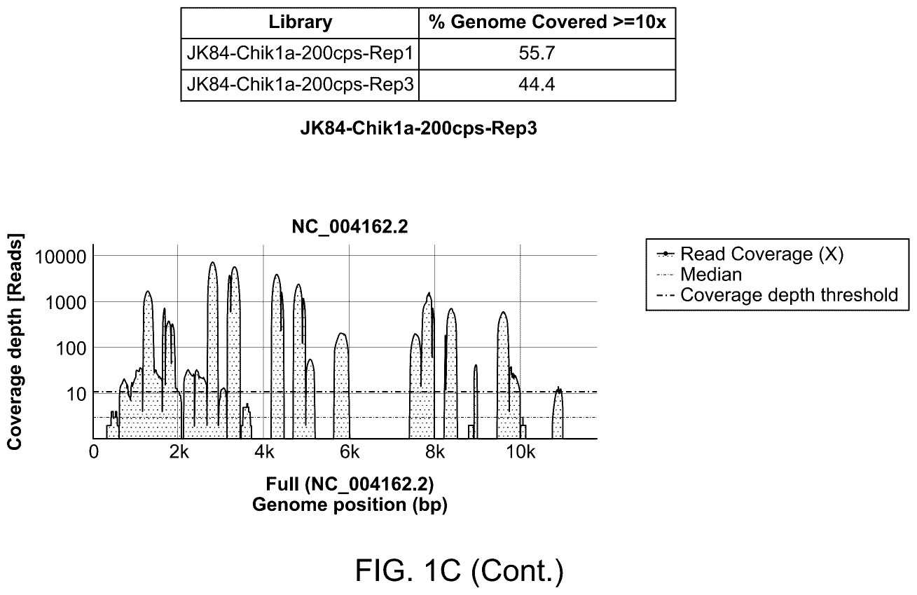

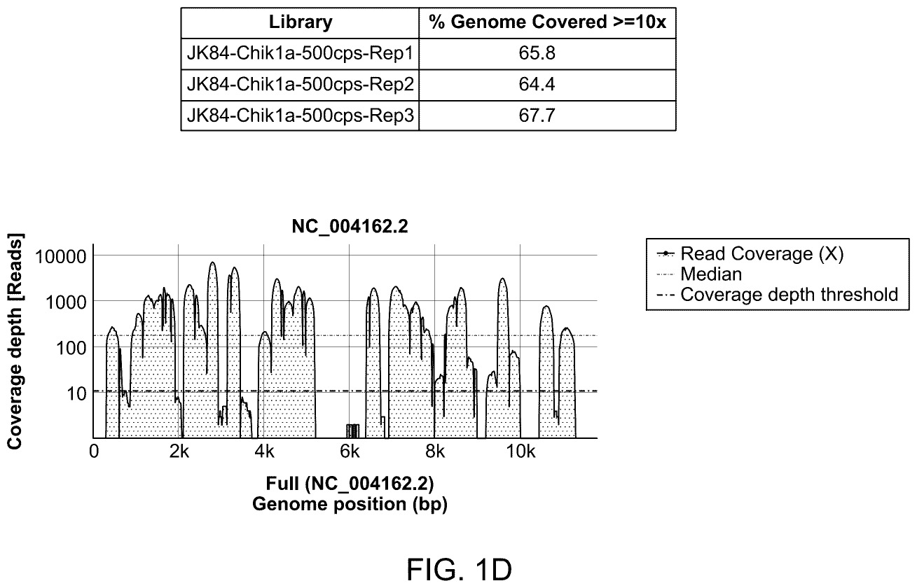

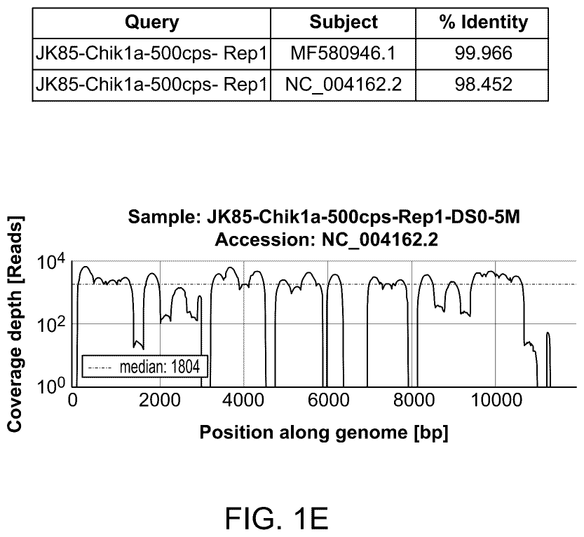

The results of these amplification and sequencing experiments are shown in FIGS. 1A-1B. FIG. 1A shows an Integrative Genomics Viewer (Robinson J T, et al. Integrative genomics viewer. Nat Biotechnol. 2011 January; 29 (1): 24-6. doi: 10.1038/nbt. 1754. PMID: 21221095; PMCID: PMC3346182.) window of genomic coverage data from 200 viral copy input amplified using the oligonucleotide primers provided in Table 1. A track for each of the three technical replicates is displayed. Genomic coverage at read depth of at least 10× for RNA input equivalent to 200 copies of the chikungunya virus genome was approximately 61%, 49.9%, and 41.4% (mean 50.8%) for the three technical replicates, respectively.

FIG. 1B shows an Integrative Genomics Viewer window of genomic coverage data from 500 viral copy input amplified using the oligonucleotide primers provided in Table 1. A track for each of the three technical replicates is displayed. Genomic coverage at read depth of at least 10× for RNA input equivalent to 500 copies of the chikungunya virus genome was approximately 82.1%, 71.8%, and 80.2% (mean 78.0%) for the three technical replicates, respectively.

FIGS. 1C-1D show the results of a comparative test using previously available oligonucleotide primers. Amplification and sequencing experiment similar to those described above were performed using a set of previously available oligonucleotide primers. In particular, FIG. 1C is an Integrative Genomics Viewer window, table, and plot of genomic coverage data from 200 viral copy input (“200 cps” in FIG. 1C) using previously available oligonucleotide primers. Two technical replicates were performed and a track for each technical replicate is displayed in the IGV window. FIG. 1D is a table and plot of genomic coverage data from 500 viral copy input using the same previously available oligonucleotide primers. Three technical replicates were performed. The previously available oligonucleotide primers used in this experiment are provided in Table 2 below. As shown in FIG. 1C, genomic coverage at read depth of at least 10× for RNA input equivalent to 200 copies of the chikungunya virus genome was approximately 55.7% and 44.4% (mean 50.1%) for the two technical replicates, respectively. As shown in FIG. 1D, genomic coverage at read depth of at least 10× for RNA input equivalent to 500 copies (cps) of the chikungunya virus genome was approximately 65.8%, 64.4%, and 67.7% (66.0%) for the three technical replicates, respectively.

TABLE 2
Previously Available Oligonucleotide Primers

| Name | Sequence | Primer Pool | SEQ ID NO: |
|---|---|---|---|
| CHIK_400_1_LEFT_3 | CTTTTTGAAGGCCCTGCAACGT | 1 | 78 |
| CHIK_400_1_RIGHT_3 | ACGGCCATCACCTCTTGTAAGT | 1 | 79 |
| CHIK_400_2_LEFT_0 | TGTCGGACAGGAAGTACCACTG | 2 | 80 |
| CHIK_400_2_RIGHT_0 | CTCATCCGCCCAATTTGTCGAG | 2 | 81 |
| CHIK_400_3_LEFT_0 | GTACTGGGTAGGGTTCGACACA | 1 | 82 |
| CHIK_400_3_RIGHT_0 | CACACGAAACCACTGTGTCACA | 1 | 83 |
| CHIK_400_4_LEFT_4 | TCGGTGTTCCATCTAAAGGGCA | 2 | 84 |
| CHIK_400_4_RIGHT_4 | CTTCATGGTGTTCGTGTTCCGT | 2 | 85 |
| CHIK_400_5_LEFT_0 | GGTGGGGCTGAACCAGAGAATA | 1 | 86 |
| CHIK_400_5_RIGHT_0 | TACAAAGCTGTCGAATTCGGCC | 1 | 87 |
| CHIK_400_6_LEFT_4 | AAGCAGAAAACACACACGGTCT | 2 | 88 |
| CHIK_400_6_RIGHT_4 | GCGCCTCTCGGAGTCTCTATTA | 2 | 89 |
| CHIK_400_7_LEFT_0 | TACAGGCAGCACAGGAAGATGT | 1 | 90 |
| CHIK_400_7_RIGHT_0 | TGGAAGTCTTCGGGCGAAATTG | 1 | 91 |
| CHIK_400_8_LEFT_1 | GGAGCAAGTGAAGACGTGTACG | 2 | 92 |
| CHIK_400_8_RIGHT_1 | TATATGGACAAGCGGGGCGAAT | 2 | 93 |
| CHIK_400_9_LEFT_0 | TGCAAGAAGGAAGAAGCTGCAG | 1 | 94 |
| CHIK_400_9_RIGHT_0 | AAGGCGATCAAGGCAAGTAACG | 1 | 95 |
| CHIK_400_10_LEFT_0 | ATCTGCACGTACGGTTGATTCG | 2 | 96 |
| CHIK_400_10_RIGHT_0 | GGTTTTGTTGAGCCCGTAGTGT | 2 | 97 |
| CHIK_400_11_LEFT_0 | GGCAAAATGCGCACTACGAATG | 1 | 98 |
| CHIK_400_11_RIGHT_0 | CCTTCCGTACGCGTTAGGAGTA | 1 | 99 |
| CHIK_400_12_LEFT_1 | AGGAGTTTACGCAGTTAGGCAAA | 2 | 100 |
| CHIK_400_12_RIGHT_1 | TTCATTCAGGGCTACTTCGGGT | 2 | 101 |
| CHIK_400_13_LEFT_0 | TGGTCCCTATCCTCGAAACAGC | 1 | 102 |
| CHIK_400_13_RIGHT_0 | TCCTGGTAGTCACGCAGATCTG | 1 | 103 |
| CHIK_400_14_LEFT_0 | TGAGGCAGCGTCCATTCTAGAA | 2 | 104 |
| CHIK_400_14_RIGHT_0 | ATACCTACCAAGTGTTGCCGGT | 2 | 105 |
| CHIK_400_15_LEFT_4 | ACCTTGCACTGCCTACTAAGAGA | 1 | 106 |
| CHIK_400_15_RIGHT_4 | ACATGGTGGTTTCAATGCTCTGG | 1 | 107 |
| CHIK_400_16_LEFT_0 | TCAGAGCATACGGTTACGCAGA | 2 | 108 |
| CHIK_400_16_RIGHT_0 | TTTGCGGTTCCTACTGGTGTTG | 2 | 109 |
| CHIK_400_17_LEFT_0 | GACGGTGTTTGCAAGGCAGTAT | 1 | 110 |
| CHIK_400_17_RIGHT_0 | TCCATGGCTGTAAAGAGGTGGT | 1 | 111 |
| CHIK_400_18_LEFT_0 | ACCTCTCCTCTCCACAGGTGTA | 2 | 112 |
| CHIK_400_18_RIGHT_0 | GCCTCTATTTGCTTTGGCCACA | 2 | 113 |
| CHIK_400_19_LEFT_0 | AAGGGACACGTTTTCACCAGAC | 1 | 114 |
| CHIK_400_19_RIGHT_0 | GGAGCATTTGACTTTTTGCACTCC | 1 | 115 |
| CHIK_400_20_LEFT_0 | CGACTTCGCATGAACCATGTCA | 2 | 116 |
| CHIK_400_20_RIGHT_0 | GTATGTATCGCCCCGTCGTCTA | 2 | 117 |
| CHIK_400_21_LEFT_0 | AGTTTGATCTAAGCGCCGATGG | 1 | 118 |
| CHIK_400_21_RIGHT_0 | AAATGTGATGGGGAACGTCTCG | 1 | 119 |
| CHIK_400_22_LEFT_0 | CGCGTGACACAGCTATTTCCTT | 2 | 120 |
| CHIK_400_22_RIGHT_0 | TGGCGTACCGACTTCTGTTGTA | 2 | 121 |
| CHIK_400_23_LEFT_0 | ACACGGACGACGAGTTATGACT | 1 | 122 |
| CHIK_400_23_RIGHT_0 | GACAATCGGACGTTGATCGGAG | 1 | 123 |
| CHIK_400_24_LEFT_0 | TAATGGCAGAGACCCCGAAAGT | 2 | 124 |
| CHIK_400_24_RIGHT_0 | TGTGTTCTGGAATGGGGAAGGT | 2 | 125 |
| CHIK_400_25_LEFT_0 | CGAGCGACATTCAATCCGTCAA | 1 | 126 |
| CHIK_400_25_RIGHT_0 | CGGCAGCAGATTATGGGTTCTT | 1 | 127 |
| CHIK_400_26_LEFT_3 | GAAGAATTTGCTGCCAGCCCTA | 2 | 128 |
| CHIK_400_26_RIGHT_3 | GTGCGGCTATAATGGCATCGAA | 2 | 129 |
| CHIK_400_27_LEFT_0 | TTAGGAGATTGAACGCCGTCCT | 1 | 130 |
| CHIK_400_27_RIGHT_0 | GTCAGACGATCTTCCAACACCC | 1 | 131 |
| CHIK_400_28_LEFT_2 | GACAGGTACGCGCTTCAAGTTC | 2 | 132 |
| CHIK_400_28_RIGHT_2 | CGCTAACGGTTTGCCCAGTTTA | 2 | 133 |
| CHIK_400_29_LEFT_0 | AGCTCCCTACTTTTGTGGAGGG | 1 | 134 |
| CHIK_400_29_RIGHT_0 | GACCGCCGTACAAGGTTATGAC | 1 | 135 |
| CHIK_400_30_LEFT_0 | TAATGTCCATGGCCACCTTTGC | 2 | 136 |
| CHIK_400_30_RIGHT_0 | TATTCTTCCGATTCTTGCGCGG | 2 | 137 |
| CHIK_400_31_LEFT_0 | CAACTTGCCCAGCTGATCTCAG | 1 | 138 |
| CHIK_400_31_RIGHT_0 | ATCGATGGTCCCCTTTACGTGT | 1 | 139 |
| CHIK_400_32_LEFT_0 | TAAGGTAACAGGTTACGCGTGC | 2 | 140 |
| CHIK_400_32_RIGHT_0 | TACGGGCTCCTTCATTAGCTCC | 2 | 141 |
| CHIK_400_33_LEFT_2 | GACCGATCTTCGACAACAAGGG | 1 | 142 |
| CHIK_400_33_RIGHT_2 | GCGTCGGGGAGAACATGTTAAG | 1 | 143 |
| CHIK_400_34_LEFT_0 | GAGAAAACCTTGCGCATGCTTG | 2 | 144 |
| CHIK_400_34_RIGHT_0 | CTGCTGGCATGTGATTGTCCAT | 2 | 145 |
| CHIK_400_35_LEFT_0 | CCAGGTTTCCTTGCAAATCGGA | 1 | 146 |
| CHIK_400_35_RIGHT_0 | GTACGTGCTGCAAGGTAGTTCC | 1 | 147 |
| CHIK_400_36_LEFT_1 | CATGTACGCACCCATTTCACCA | 2 | 148 |
| CHIK_400_36_RIGHT_1 | GGTCCCCGAATTCAGCATTACG | 2 | 149 |
| CHIK_400_37_LEFT_0 | CTGCAAGGTCGATCAATGCCAT | 1 | 150 |
| CHIK_400_37_RIGHT_0 | TCATTGTTACCCCACGTGACCT | 1 | 151 |
| CHIK_400_38_LEFT_0 | AGTGGGTGACGCATAAGAAGGA | 2 | 152 |
| CHIK_400_38_RIGHT_0 | TGGTGTCAGTTCGTACGGTGTA | 2 | 153 |
| CHIK_400_39_LEFT_0 | ATACTCCTGTCGATGGTGGGTG | 1 | 154 |
| CHIK_400_39_RIGHT_0 | CGGGATCACTGTTACGTGTTCG | 1 | 155 |
| CHIK_400_40_LEFT_0 | TTTTTAGCCGTACTGAGCGTCG | 2 | 156 |
| CHIK_400_40_RIGHT_0 | ATTTTCGGTGTCGCAGAAGCAG | 2 | 157 |
| CHIK_400_41_LEFT_0 | GCGGTACAGCAGAGTGTAAGGA | 1 | 158 |
| CHIK_400_41_RIGHT_0 | GTCGCCTTTGTACACCACGATT | 1 | 159 |
| CHIK_400_42_LEFT_0 | CCATGCCGTCACAGTTAAGGAC | 2 | 160 |
| CHIK_400_42_RIGHT_0 | AGTTCATCGCTCTTACCGGGTT | 2 | 161 |
| CHIK_400_43_LEFT_0 | ATTGGCTAAAAGAACGAGGGGC | 1 | 162 |
| CHIK_400_43_RIGHT_0 | TTCCCGAATAGTGACGGCGTTA | 1 | 163 |
| CHIK_400_44_LEFT_0 | GCACCCATTCCTCAGACTTTGG | 2 | 164 |
| CHIK_400_44_RIGHT_0 | CCTGCTAAACGACACGCATAGC | 2 | 165 |

To determine if there is a difference between the genome sequence of the viral material sequenced in the amplification and sequencing experiments and the chikungunya virus reference genome sequence NC_004162.2 (SEQ ID NO: 1) that was used for primer design, a consensus genome was generated from the sequencing reads using the DRAGEN Microbial Enrichment workflow. This consensus genome was aligned to the chikungunya virus reference genome sequence NC_004162.2 using the NCBI BLAST tool, using Nucleotide BLAST (blastn) against the nr/nt database with default parameters. As shown in the table of FIG. 1E, BLAST alignment results show that the chikungunya genome with the greatest sequence identity is not genome NC_004162.2 (98.45% identity), which was used for primer design, but rather genome MF580946.1 (99.97%). A plot of genomic sequencing coverage for alignment to NCBI Reference Sequence NC_004162.2 is shown in FIG. 1E.

These results demonstrate that the sets of oligonucleotide primers provided in Table 1 can successfully amplify the chikungunya virus genome for downstream sequencing resulting in high genomic coverage. Compared to the previously available oligonucleotide primers tested, the oligonucleotide primers provided in Table 1 result in increased sequencing coverage of the chikungunya virus genome at 500 viral copy input. Further, the sets of oligonucleotide primers provided in Table 1 can provide an amplicon sequencing library that identifies variants in the chikungunya virus genome and is useful for distinguishing between strains of the chikungunya virus genome.

### Example 2: Amplification and Sequencing of the Chikungunya Virus Genome with High-Copy Nucleic Acid Input

Oligonucleotide primers listed in Table 1 were computationally designed using a software tool with NCBI Reference Sequence NC_004162.2 as the template genome for primer design. After primer design, DNA oligonucleotides were synthesized according to the design, normalized to 100 μM, pooled into two primer pools as described in Table 1, and diluted to 10 μM for each pool to generate two overlapping sets of amplicons. RNA input equivalent to 200 and 500 copies of the chikungunya virus genome was used for two separate amplification and sequencing experiments. After amplification, amplicon libraries were denatured and diluted from a pooled library according to the Illumina® NextSeq® 500 and 550 Sequencing Systems Denature and Dilute Libraries Guide (15048776). Libraries were sequenced on the Illumina® NextSeq® 550 instrument at 2×149 base pair read length, unless stated otherwise, and normalized to 1M paired-end read depth based on current sequencing recommendations. Analysis was executed using the DRAGEN® Viral Lineage App v0.4.0.

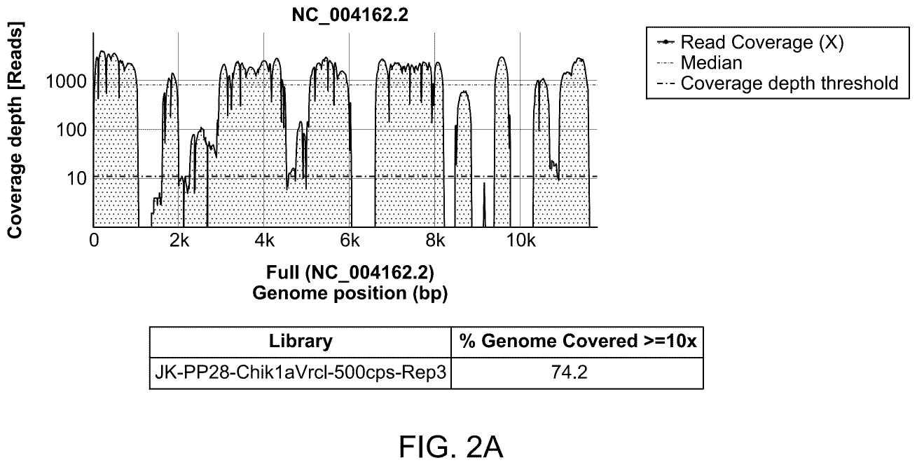

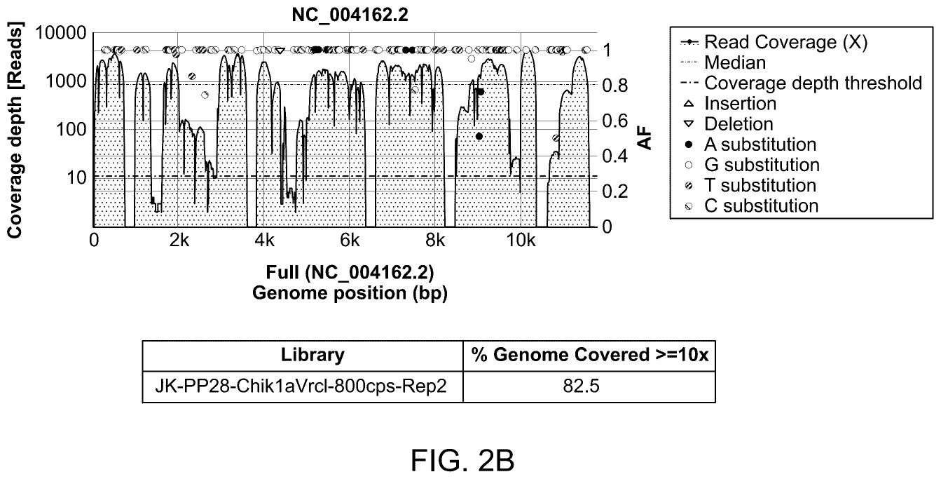

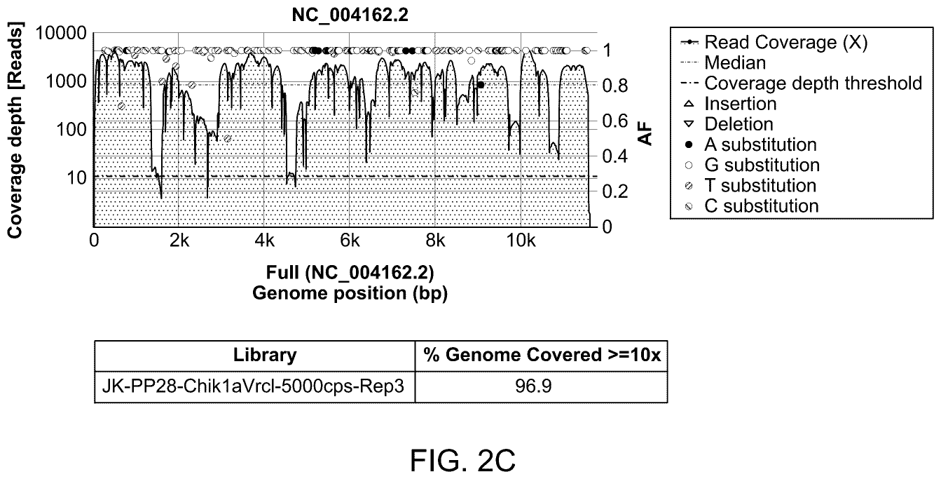

The results of these amplification and sequencing experiments are shown in FIGS. 2A-2C. FIG. 2A shows a plot of coverage data from 500 viral copy input. Genomic coverage at read depth of at least 10× for RNA input equivalent to 500 copies of the chikungunya virus genome was approximately 74.2%. FIG. 2B shows a plot of coverage data from 800 viral copy input. Genomic coverage at read depth of at least 10× for RNA input equivalent to 800 copies of the chikungunya virus genome was approximately 82.5%. FIG. 2C shows a plot of coverage data from 5000 viral copy input. Genomic coverage at read depth of at least 10× for RNA input equivalent to 5000 copies of the chikungunya virus genome was approximately 96.9%.

These results demonstrate that the sets of oligonucleotide primers provided in Table 1 can successfully amplify the chikungunya virus genome for downstream sequencing resulting in high genomic coverage. Further, increasing the viral RNA input for the amplification and sequencing workflow results in increased genomic coverage, with complete genomic coverage achieved at 5000 viral copy input using the oligonucleotide primers provided in Table 1.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of characterizing a chikungunya virus genome, the method comprising:
obtaining a sample comprising a nucleic acid of viral origin;
obtaining a first plurality of oligonucleotide primers comprising sequences selected from the group consisting of SEQ ID NOs 2, 4, 5, 8, 9, 12, 13, 16, 17, 20, 21, 24, 25, 28, 29, 32, 33, 36, 37, 40, 41, 44, 45, 48, 49, 52, 53, 56, 57, 60, 61, 64, 65, 68, 69, 72, 73, and 76;
obtaining a second plurality of oligonucleotide primers comprising sequences selected from the group consisting of SEQ ID NOs 3, 6, 7, 10, 11, 14, 15, 18, 19, 22, 23, 26, 27, 30, 31, 34, 35, 38, 39, 42, 43, 46, 47, 50, 51, 54, 55, 58, 59, 62, 63, 66, 67, 70, 71, 74, 75, and 77;
amplifying the nucleic acid using the first and the second pluralities of oligonucleotide primers to produce first and second amplification products;
sequencing the first and the second amplification products to produce sequencing reads;
aligning the sequencing reads to a reference chikungunya genome to produce an alignment; and
based on the alignment, identifying one or more variants in the chikungunya virus genome compared to the reference chikungunya virus genome.

2. The method of claim 1, wherein the nucleic acid is RNA.

3. The method of claim 2, further comprising, before the amplification step, reverse transcribing the RNA to produce cDNA.

4. The method of claim 1, wherein the first plurality of oligonucleotide primers are configured for use as pairs, wherein the pairs are selected from the group consisting of SEQ ID NOs: 2 and 4, 5 and 8, 9 and 12, 13 and 16, 17 and 20, 21 and 24, 25 and 28, 29 and 32, 33 and 36, 37 and 40, 41 and 44, 45 and 48, 49 and 52, 53 and 56, 57 and 60, 61 and 64, 65 and 68, 69 and 72, and 73 and 76; and
the second plurality of oligonucleotide primers are configured for use as pairs, wherein the pairs are selected from the group consisting of SEQ ID NOs: 3 and 6, 7 and 10, 11 and 14, 15 and 18, 19 and 22, 23 and 26, 27 and 30, 31 and 34, 35 and 38, 39 and 42, 43 and 46, 47 and 50, 51 and 54, 55 and 58, 59 and 62, 63 and 66, 67 and 70, 71 and 74, and 75 and 77.

5. The method of claim 1, wherein the reference chikungunya genome is about 80%, 85%, 90%, 95%, 96%, 87%, 98%, 99%, or 100% identical to SEQ ID NO: 1.

6. The method of claim 1, wherein the sequencing is next generation sequencing (NGS).

7. The method of claim 1, wherein the nucleic acid is obtained from a biological sample.

8. The method of claim 7, wherein the sample comprises blood or serum.

9. The method of claim 1, further comprising, after the alignment step, quantifying a genomic coverage of the sequencing reads.

10. The method of claim 9, wherein the genomic coverage is at least 50%, 55%, 60%, 65%, 70%, 75%, 80%, 85%, 95%, 96%, 97%, 98%, 99%, or 100% of the reference chikungunya genome at a minimum read depth of at least ten reads.

11. The method of claim 1, wherein the RNA is present in the sample in an amount equivalent to 200 copies of the chikungunya virus genome.

12. The method of claim 11, wherein the genomic coverage is at least 50% of the reference chikungunya genome at a minimum read depth of at least ten reads.

13. The method of claim 1, wherein the RNA is present in the sample in an amount equivalent to 500 copies of the chikungunya virus genome.

14. The method of claim 13, wherein the genomic coverage is at least 75% of the reference chikungunya genome at a minimum read depth of at least ten reads.

15. The method of claim 1, wherein the RNA is present in the sample in an amount equivalent to 800 copies of the chikungunya virus genome.

16. The method of claim 15, wherein the genomic coverage is at least 80% of the reference chikungunya genome at a minimum read depth of at least ten reads.

17. The method of claim 1, wherein the RNA is present in the sample in an amount equivalent to 5000 copies of the chikungunya virus genome.

18. The method of claim 17, wherein the genomic coverage is at least 95% of the reference chikungunya genome at a minimum read depth of at least ten reads.

19. The method of claim 1, further comprising distinguishing the chikungunya virus genome from a second nucleic acid of viral origin based on the identified variants.

20. A method of detecting chikungunya virus RNA in a sample, the method comprising:
obtaining a sample;
isolating RNA from the sample;
obtaining a first plurality of oligonucleotide primers comprising sequences selected from the group consisting of SEQ ID NOs 2, 4, 5, 8, 9, 12, 13, 16, 17, 20, 21, 24, 25, 28, 29, 32, 33, 36, 37, 40, 41, 44, 45, 48, 49, 52, 53, 56, 57, 60, 61, 64, 65, 68, 69, 72, 73, and 76;
obtaining a second plurality of oligonucleotide primers comprising sequences selected from the group consisting of SEQ ID NOs 3, 6, 7, 10, 11, 14, 15, 18, 19, 22, 23, 26, 27, 30, 31, 34, 35, 38, 39, 42, 43, 46, 47, 50, 51, 54, 55, 58, 59, 62, 63, 66, 67, 70, 71, 74, 75, and 77;
reverse transcribing the RNA to produce cDNA
amplifying the cDNA using the first and the second pluralities of oligonucleotide primers to produce first and second amplification products;
sequencing the first and the second amplification products to produce sequencing reads;
quantifying the sequencing reads; and
determining, based on the quantity of sequencing reads, a presence or absence of chikungunya virus RNA in the sample.

21. The method of claim 20, wherein the sample is a biological sample.

22. The method of claim 21, wherein the biological sample comprises blood or serum.

23. The method of claim 20, wherein the sample is an environmental sample.

24. The method of claim 23, wherein the environmental sample comprises an extract from one or more mosquitos, or a wastewater or air filter sample.

25. The method of claim 20, wherein the first plurality of oligonucleotide primers are configured for use as pairs, wherein the pairs are selected from the group consisting of SEQ ID NOs: 2 and 4, 5 and 8, 9 and 12, 13 and 16, 17 and 20, 21 and 24, 25 and 28, 29 and 32, 33 and 36, 37 and 40, 41 and 44, 45 and 48, 49 and 52, 53 and 56, 57 and 60, 61 and 64, 65 and 68, 69 and 72, and 73 and 76; and
the second plurality of oligonucleotide primers are configured for use as pairs, wherein the pairs are selected from the group consisting of SEQ ID NOs: 3 and 6, 7 and 10, 11 and 14, 15 and 18, 19 and 22, 23 and 26, 27 and 30, 31 and 34, 35 and 38, 39 and 42, 43 and 46, 47 and 50, 51 and 54, 55 and 58, 59 and 62, 63 and 66, 67 and 70, 71 and 74, and 75 and 77.

26. The method of claim 20, wherein the sequencing is next generation sequencing (NGS).

27. The method of claim 20, further comprising assembling the sequencing reads to produce a consensus sequence.

28. The method of claim 27, wherein the consensus sequence is produced if at least 35 amplicons are detected in the sequencing reads.

29. A kit comprising:
one or more buffers;
a reverse transcriptase;
a first plurality of oligonucleotide primers comprising sequences selected from the group consisting of SEQ ID NOs 2, 4, 5, 8, 9, 12, 13, 16, 17, 20, 21, 24, 25, 28, 29, 32, 33, 36, 37, 40, 41, 44, 45, 48, 49, 52, 53, 56, 57, 60, 61, 64, 65, 68, 69, 72, 73, and 76;
a second plurality of oligonucleotide primers comprising sequences selected from the group consisting of SEQ ID NOs 3, 6, 7, 10, 11, 14, 15, 18, 19, 22, 23, 26, 27, 30, 31, 34, 35, 38, 39, 42, 43, 46, 47, 50, 51, 54, 55, 58, 59, 62, 63, 66, 67, 70, 71, 74, 75, and 77;
a DNA polymerase; and
one or more library preparation agents.


SEQUENCE LISTING (165 sequences)

SEQ ID NO: 1 - RNA, 11826 nt, Chikungunya virus
  source 1..11826: mol_type = other RNA
atggctgcgtgagacacacgtagcctaccagtttcttactgctctactctgcaaagcaagagattaagaacccatcatggatcctgtgtacgtggacatagacgctgacagcgcctttttgaaggccctgcaacgtgcgtaccccatgtttgaggtggaacctaggcaggtcacaccgaatgaccatgctaatgctagagcgttctcgcatctagctataaaactaatagagcaggaaattgatcccgactcaaccatcctggatattggtagtgcgccagcaaggaggatgatgtcggacaggaagtaccactgcgtttgcccgatgcgcagtgcagaagatcccgagagactcgccaattatgcgagaaagctagcatctgccgcaggaaaagtcctggacagaaacatctctggaaagatcggggacttacaagcagtaatggccgtgccagacacggagacgccaacattctgcttacacacagatgtatcatgtagacagagagcagacgtcgcgatataccaagacgtctatgctgtacacgcacccacgtcgctataccaccaggcgattaaaggggtccgattggcgtactgggtagggtttgacacaaccccgttcatgtacaatgccatggcgggtgcctacccctcatactcgacaaattgggcagatgagcaggtactgaaggctaagaacataggattatgttcaacagacctgacggaaggtagacgaggcaaattgtctattatgagaggaaaaaagctagaaccgtgcgaccgtgtgctgttctcagtagggtcaacgctctacccggaaagccgtaagctacttaagagctggcacctaccatcggtgttccatttaaagggcaagctcagcttcacatgccgctgtgatacagtggtttcgtgcgaaggctacgtcgttaagagaataacgatgagcccaggcctttacggaaaaaccacagggtatgcggtaacccaccacgcagacggattcctgatgtgcaagaccaccgacacggttgacggcgaaagagtgtcattctcggtgtgcacgtacgtgccggcgaccatttgtgatcaaatgaccggcatccttgctacagaagtcacgccggaggatgcacagaagctgttggtggggctgaaccagagaatagtggttaacggcagaacgcaacggaatacgaacaccatgaaaaactatatgattcccgtggtcgcccaagccttcagtaagtgggcaaaggagtgccggaaagacatggaagatgaaaaactcctgggggtcagagaaagaacactgacctgctgctgtctatgggcatttaagaagcagaaaacacacacggtctacaagaggcctgatacccagtcaattcagaaggttcaggccgagtttgacagctttgtggtaccgagcctgtggtcgtccgggttgtcaatcccgttgaggactagaatcaaatggttgttaagcaaggtgccaaaaaccgacctgaccccatacagcggggacgcccaagaagcccgggacgcagaaaaagaagcagaggaagaacgagaagcagaactgactcttgaagccctaccaccccttcaggcagcacaggaagatgttcaggtcgaaatcgacgtggaacagcttgaggacagagcgggtgcaggaataatagagactccgagaggagctatcaaagttactgcccaaccaacagaccacgtcgtgggagagtacttggttctttccccgcagaccgtactacgtagccaaaagcttagcctgattcacgctttggcggagcaagtgaagacgtgcacgcacagcggacgagcagggaggtatgcggtcgaagcgtacgacggcagagtcctagtgccctcaggctacgcaatctcgcctgaagacttccagagcctaagcgaaagcgcaacgatggtgtacaacgaaagagagttcgtaaacagaaagctacaccatattgcgatgcatggaccagccctgaacaccgacgaagagtcgtatgagctggtgagggcagagaggacagaacacgagtacgtctacgacgtggaccagagaagatgctgtaagaaggaagaagctgcaggactggtactggtgggcgacttgactaatccgccctaccacgaattcgcatatgaagggctaaaaatccgccctgcctgcccatacaaaattgcagtcataggagtcttcggagtaccaggatctggcaagtcagctattatcaagaacctagttaccaggcaagacctggtgactagcggaaagaaagaaaactgccaagaaatcaccaccgacgtgatgagacagagaggtctagagatatctgcacgtacggttgactcgctgctcttgaatggatgtaacagaccagtcgacgtgttgtacgtagacgaggcgtttgcgtgccactctggaacgttacttgcattgatcgccttggtgagaccaagacagaaagttgtactttgtggtgacccgaagcagtgcggcttcttcaatatgatgcagatgaaagtcaactataatcacaacatctgcacccaagtgtaccacaaaagtatctccaggcggtgtacactgcctgtgactgccattgtgtcatcgttgcattacgaaggcaaaatgcgcactacgaatgagtacaacaagccgattgtagtggacactacaggctcaacaaaacctgaccctggagatctcgtgttaacgtgcttcagaggatgggttaaacaactgcaaattgactatcgtggacacgaggtcatgacagcagccgcatcccaagggttaaccagaaaaggagtttacgcagttaggcaaaaagttaacgaaaacccgctttatgcatcaacgtcagagcacgtcaacgtactcctaacgcgtacggaaggtaaactggtatggaagacactctccggtgacccgtggataaagacgctgcagaacccaccgaaaggaaacttcaaagcaactattaaggagtgggaggtggagcatgcatcaataatggcgggcatctgcagtcaccaaatgacctttgatacattccaaaacaaagccaacgtttgttgggctaagagtttggtccctatcctcgaaacagcggggataaaactaaacgacaggcagtggtcccagataattcaagccttcaaagaagacaaagcatattcacccgaagtagccctgaatgaaatatgcacgcgcatgtatggggtggatctagacagcgggctattttctaaaccgttggtgtctgtgtattacgcggataaccactgggataataggcctggagggaagatgttcggattcaaccccgaggcagcatccattctagaaagaaagtatccatttacaaaagggaagtggaacatcaacaagcagatctgcgtgactaccaggaggatagaagacttcaaccctaccaccaacattataccggccaacaggagactaccacactcattagtggccgaacaccgcccagtaaaaggggaaagaatggaatggctggttaacaagataaacggccaccacgtgctcctggtcagtggctgtagccttgcactgcctactaagagagtcacttgggtagcgccactaggtgtccgcggagcggactatacatacaacctagagttgggtctgccagcaacgcttggtaggtatgacctagtggtcataaacatccacacaccttttcgcatacaccattatcaacagtgcgtagaccacgcaatgaaactgcaaatgctcgggggtgactcattgagactgctcaaaccgggtggctctctattgatcagagcatatggttacgcagatagaaccagtgaacgagtcatctgcgtattgggacgcaagtttagatcatctagagcgttgaaaccaccatgtgtcaccagcaacactgagatgttttttctattcagcaactttgacaatggcagaaggaatttcacaactcatgtcatgaacaatcaactgaatgcagcctttgtaggacaggccacccgagcaggatgtgcaccgtcgtaccgggtaaaacgcatggatatcgcgaagaacgatgaagagtgcgtagtcaacgccgccaaccctcgcgggttaccaggtgacggtgtttgcaaggcagtatacaaaaaatggccggagtcctttaagaacagtgcaacaccagtgggaaccgcaaaaacagtcatgtgcggtacgtatccagtaatccacgccgttggaccaaacttctctaattattcggagtctgaaggggaccgagaattggcggctgcctatcgagaagtcgcaaaggaggtaactagactgggagtaaatagtgtagctatacctctcctctccacaggtgtatactcaggagggaaagacaggctgacccagtcactgaaccacctctttacagccatggactcgacggatgcagacgtggtcatctactgccgcgacaaagaatgggagaagaaaatatctgaggccatacagatgcggacccaagtggagctgctggatgagcacatctccatagactgcgatgttgttcgcgtgcaccctgacagcagcttggcaggcagaaaaggatacagcaccacggaaggcgcactgtactcatatctagaagggacccgttttcaccaaacggcagtggatatggcagagatatatactatgtggccaaagcaaacagaggccaacgagcaagtttgcctatatgccctgggggaaagtattgaatcgatcaggcagaaatgcccggtggatgatgcagatgcatcatctcccccgaaaactgtcccgtgcctctgccgttacgccatgacaccagaacgcgttacccgacttcgcatgaaccatgtcacaagcataattgtgtgttcttcgtttccccttccaaagtacaaaatagaaggagtgcaaaaagtcaaatgctccaaggtaatgctatttgaccacaacgtgccatcgcgcgtaagtccaagggaatacagaccttcccaggagtctgtacaggaagcgagtacgaccacgtcactgacgcatagccaattcgatctaagcgttgacggcaagatactgcccgtcccgtcagacctggatgctgacgccccagccctagaaccagcccttgacgacggggcgatacacacgttgccatctgcaaccggaaaccttgcggccgtgtctgactgggtaatgagcaccgtacctgtcgcgccgcccagaagaaggcgagggagaaacctgactgtgacatgcgacgagagagaagggaatataacacccatggctagcgtccgattctttagggcagagctgtgtccagtcgtacaagaaacagcggagacgcgtgacacagctatgtctcttcaggcaccgccgagtaccgccacggaactgagtcacccgccgatctccttcggtgcaccaagcgagacgttccccatcacatttggggacttcaacgaaggagaaatcgaaagcttgtcttctgagctactaactttcggagacttcctacccggagaagtggatgatttgacagatagcgactggtccacgtgctcagacacggacgacgagttacgactagacagggcaggtgggtatatattctcgtcggacactggtccaggtcatttacaacagaagtcagtacgccagtcagtgctgccggtgaacaccctggaggaagtccacgaggagaagtgttacccacctaagctggatgaagcaaaggagcaactactacttaagaaactccaggagagtgcatccatggccaacagaagcaggtatcagtcgcgcaaagtagaaaacatgaaagcaacaatcatccagagactaaagagaggctgtagattatacttaatgtcagagaccccaaaagtccctacctaccggaccacatatccggcgcctgtgtactcgcctccgattaacgtccgactgtccaaccccgagtccgcagtggcagcatgcaatgagttcttggctagaaactatccaactgtttcatcataccaaatcaccgacgagtatgatgcatatctagacatggtggacgggtcggagagttgtctggaccgagcgacattcaatccgtcaaaacttaggagctacccaaaacagcacgcttaccacgcgccctccatcagaagcgctgtaccgtccccattccagaacacactacagaatgtactggcagcagccacgaaaagaaactgcaacgtcacacagatgagggaattacccactttggactcagcagtattcaacgtggagtgtttcaaaaaattcgcatgcaaccaagaatactgggaagaatttgctgccagccctatcaggataacaactgagaatttaacaacctatgttactaaactaaaggggccaaaagcagcagcgctatttgcaaaaacccataatctgctgccactgcaggaagtgccaatggataggttcacagtagacatgaaaagggatgtgaaggtgactcctggtacaaagcacacagaggaaagacctaaggtacaggttatacaggcggctgaacccttggcaacagcatacctatgtgggattcacagagagctggttaggaggctgaacgccgtcctcctacccaatgtacatacactatttgacatgtctgccgaggatttcgatgccatcatagccgcacactttaagccaggagacactgttttagaaacggacatagcctcctttgataagagccaagatgattcacttgcgcttactgctttaatgctgttagaggatttaggggtggatcactccctgttggacttgatagaggctgctttcggagagatttccagctgtcatctaccgacaggtacgcgcttcaagttcggcgccatgatgaaatctggtatgttcctaactctgttcgtcaacacactgctaaatatcaccatcgccagccgagtgctggaagatcgtctgacaaaatccgcgtgcgcagccttcatcggcgacgacaacataatacatggagtcgtctccgatgaattgatggcagccagatgcgccacttggatgaacatggaagtgaagatcatagatgcagttgtatcccagaaagccccttacttttgtggagggtttatactgcacgatatcgtgacaggaacagcttgcagagtggcagacccgctaaaaaggctatttaaactgggcaaaccgctagcggcaggtgacgaacaagatgaggatagaagacgagcgctggctgacgaagtggtcagatggcaacgaacagggctaattgatgagttggagaaagcggtatactctaggtatgaagtgcagggtatatcagttgtggtaatgtccatggccacctttgcaagctccagatccaacttcgagaagctcagaggacccgtcgtaactttgtacggcggtcctaaataggtacgcactacagctacctattttgcagaagccgacagtaagtacctaaacactaatcagctacaatggagttcatcccaacccaaactttttacaacaggaggtaccagcctcgaccctggactccgcgccctactatccaagtcatcaggcccagaccgcgcccgcagaggcaagctgggcaacttgcccagctgatctcagcagttaataaactgacaatgcgcgcggtaccccaacagaagccacgcaagaatcggaagaataagaagcaaaagcaaaagcagcaggcgccacaaaacaacacaaaccaaaagaagcagccacctaaaaagaaaccagctcaaaagaaaaagaagccgggccgcagagagaggatgtgcatgaaaatcgaaaatgactgtattttcgaagtcaagcacgaaggtaaggtaacaggttacgcgtgcttggtgggggacaaagtaatgaaaccagcacacgtaaaggggaccatcgataacgcggacctggccaaattggcctttaagcggtcatctaagtacgaccttgaatgcgcgcagatacccgtgcacatgaagtccgacgcttcgaagttcacccatgagaaaccggaggggtactacaactggcaccacggagcagtacagtactcaggaggccggttcaccatccctacaggtgcgggcaaaccaggggacagcggtagaccgatcttcgacaacaagggacgcgtggtggccatagtcttaggaggagctaatgaaggagcccgtacagccctctcagtggtgacctggaataaagacattgtcactaaaatcacccctgagggagccgaagagtggagtcttgccatcccagttatgtgcctgttggcaaataccacgttcccctgctcccagcccccttgcataccctgctgctacgaaaaggaaccggaggaaaccctacgcatgcttgaggacaacgtcatgagacctgggtactatcagctgctacaagcatcattaacatgttctccccaccgccagcgacgcagcaccaaggacaacttcaatgtctataaagccacaagaccatacctagctcactgtcccgactgtggagaagggcactcgtgccatagtcccgtagcactagaacgcatcagaaatgaagcgacagacgggacgctgaaaatccaggtctccttgcaaattggaatagggacggatgatagccatgattggaccaagctgcgttacatggacaatcacataccagcagacgcagggagggccgggctatttgtaagaacatcagcaccatgcacgattactggaacaatgggacacttcatcctggcccgatgtccgaaaggagaaactctgacggtgggattcactgacagtaggaagattagtcactcatgtacgcacccatttcaccacgaccctcctgtgataggccgggaaaaattccattcccgaccgcagcacggtaaagagctaccttgcagcacgtacgtgcagagcaacgccgcaactgccgaggagatagaggtacacatgcccccagacacccctgatcgcacattgctgtcacaacagtccggcaacgtaaagatcacagtcaatagtcagacggtgcggtataagtgtaattgcggtggctcaaatgaaggactaataactacagataaagtgattaataactgcaaggttgatcaatgtcatgccgcggtcaccaatcacaaaaagtggcagtataactcccctctggtcccgcgtaacgctgaactcggggaccgaaaaggaaaaattcacatcccgtttccgctggcaaatgtaacatgcatggtgcctaaagcaaggaaccccaccgtgacgtacgggaaaaaccaagtcatcatgctactgtatcctgaccacccaacactcctgtcctaccggagtatgggagaagaaccaaactatcaagaagagtgggtgacgcacaagaaggaggtcgtgctaaccgtgccgactgaagggctcgaggttacgtggggcaacaacgagccgtataagtattggccgcagttatctgcaaacggtacagcccacggccacccgcatgagataatcttgtactattatgagctgtaccctactatgactgtagtagttgtgtcagtggcctcgttcatactcctgtcgatggtgggtatggcagtggggatgtgcatgtgtgcacgacgcagatgcatcacaccatacgaactgacaccaggagctaccgtccctttcctgcttagcctaatatgctgcatcagaacagctaaagcggccacataccaagaggctgcggtatacctgtggaacgagcagcaacctttgttttggctacaagcccttattccgctggcagccctgattgtcctatgcaactgtctgagactcttaccatgctgttgtaaaacgttggcttttttagccgtaatgagcatcggtgcccacactgtgagcgcgtacgaacacgtaacagtgatcccgaacacggtgggagtaccgtataagactctagtcaacagaccgggctacagccccatggtactggagatggagctactgtcagtcactttggagccaacgctatcgcttgattacatcacgtgcgaatacaaaaccgtcatcccgtctccgtacgtgaaatgctgcggtacagcagagtgcaaggacaaaaacctacctgactacagctgtaaggtcttcaccggcgtctacccatttatgtggggcggcgcctactgcttctgcgacgctgaaaacacgcaattgagcgaagcacatgtggagaagtccgaatcatgcaaaacagaatttgcatcagcatacagggctcataccgcatccgcatcagctaagctccgcgtcctttaccaaggaaataacatcactgtaactgcctatgcaaacggcgaccatgccgtcacagttaaggacgccaaattcattgtggggccaatgtcttcagcctggacaccttttgacaacaaaatcgtggtgtacaaaggtgacgtttacaacatggactacccgccctttggcgcaggaagaccaggacaatttggcgatatccaaagtcgcacgcctgagagcaaagacgtctatgctaacacacaactggtactgcagagaccggctgcgggtacggtacacgtgccatactctcaggcaccatctggctttaagtattggttaaaagaacgaggggcgtcgctacagcacacagcaccatttggctgccaaatagcaacaaacccggtaagagcgatgaactgcgccgtagggaacatgcccatctccatcgacataccggatgcggccttcactagggtcgtcgacgcgccctctttaacggacatgtcatgcgaggtaccagcctgcacccattcctcagactttgggggcgtcgccattattaaatatgcagtcagcaagaaaggcaagtgtgcggtgcattcgatgaccaacgccgtcactatccgggaagctgagatagaagttgaagggaattctcagctgcaaatctctttctcgacggccttggccagcgccgaattccgcgtacaagtctgttctacacaagtacactgtgcagccgagtgccaccctccgaaggaccacatagtcaactacccggcgtcacataccaccctcggggtccaggacatttccgctacggcgatgtcatgggtgcagaagatcacgggaggtgtgggactggttgtcgctgttgcagcactgattctaatcgtggtgctatgcgtgtcgttcagcaggcactaacttgacgactaagcatgaaggtatatgtgtcccctaagagacacaccgtatatagctaataatctgtagatcaaagggctatataacccctgaatagtaacaaaatacaaaatcactaaaaattataaaaaaaaaaaaaaaaaaacagaaaaatatataaataggtatacgtgtcccctaagagacacattgtatgtaggtgataagtatagatcaaagggccgaacaacccctgaatagtaacaaaatataaaaattaataaaaatcataaaatagaaaaaccataaacagaagtagttcaaagggctataaaaacccctgaatagtaacaaaacataaaactaataaaaatcaaatgaataccataattggcaaacggaagagatgtaggtacttaagcttcctaaaagcagccgaactcactttgagatgtaggcatagcataccgaactcttccacgattctccgaacccacagggacgtaggagatgttattttgtttttaatatttc

SEQ ID NO: 2 - DNA, 22 bp, synthetic construct
  source 1..22: mol_type = other DNA
agacacacgtagcctaccagtt

SEQ ID NO: 3 - DNA, 22 bp, synthetic construct
  source 1..22: mol_type = other DNA
tgcagaagatcccgagagactc

SEQ ID NO: 4 - DNA, 23 bp, synthetic construct
  source 1..23: mol_type = other DNA
tccagagatgtttctgtccagga

SEQ ID NO: 5 - DNA, 22 bp, synthetic construct
  source 1..22: mol_type = other DNA
gcctacccctcatactcgacaa

SEQ ID NO: 6 - DNA, 22 bp, synthetic construct
  source 1..22: mol_type = other DNA
taccttccgtcaggtctgttga

SEQ ID NO: 7 - DNA, 22 bp, synthetic construct
  source 1..22: mol_type = other DNA
aggcctttacggaaaaaccaca

SEQ ID NO: 8 - DNA, 22 bp, synthetic construct
  source 1..22: mol_type = other DNA
aatgacactctttcgccgtcaa

SEQ ID NO: 9 - DNA, 22 bp, synthetic construct
  source 1..22: mol_type = other DNA
aaaactcctgggggtcagagaa

SEQ ID NO: 10 - DNA, 22 bp, synthetic construct
  source 1..22: mol_type = other DNA
agaccgtgtgtgttttctgctt

SEQ ID NO: 11 - DNA, 20 bp, synthetic construct
  source 1..20: mol_type = other DNA
ttgaagccctaccacccctt

SEQ ID NO: 12 - DNA, 22 bp, synthetic construct
  source 1..22: mol_type = other DNA
tattattcctgcacccgctctg

SEQ ID NO: 13 - DNA, 22 bp, synthetic construct
  source 1..22: mol_type = other DNA
gaagacttccagagcctaagcg

SEQ ID NO: 14 - DNA, 22 bp, synthetic construct
  source 1..22: mol_type = other DNA
tccatgcatcgcaatatggtgt

SEQ ID NO: 15 - DNA, 22 bp, synthetic construct
  source 1..22: mol_type = other DNA
caggatctggcaagtcagctat

SEQ ID NO: 16 - DNA, 22 bp, synthetic construct
  source 1..22: mol_type = other DNA
tggcagttttctttctttccgc

SEQ ID NO: 17 - DNA, 25 bp, synthetic construct
  source 1..25: mol_type = other DNA
actataatcacaacatctgcaccca

SEQ ID NO: 18 - DNA, 22 bp, synthetic construct
  source 1..22: mol_type = other DNA
atgacacaatggcagtcacagg

SEQ ID NO: 19 - DNA, 22 bp, synthetic construct
  source 1..22: mol_type = other DNA
ccgctttatgcatcaacgtcag

SEQ ID NO: 20 - DNA, 22 bp, synthetic construct
  source 1..22: mol_type = other DNA
ccggagagtgtcttccatacca

SEQ ID NO: 21 - DNA, 24 bp, synthetic construct
  source 1..24: mol_type = other DNA
caagccttcaaagaagacaaagca

SEQ ID NO: 22 - DNA, 22 bp, synthetic construct
  source 1..22: mol_type = other DNA
aatagcccgctgtctagatcca

SEQ ID NO: 23 - DNA, 22 bp, synthetic construct
  source 1..22: mol_type = other DNA
cactcattagtggccgaacacc

SEQ ID NO: 24 - DNA, 20 bp, synthetic construct
  source 1..20: mol_type = other DNA
cacgtggtggccgtttatct

SEQ ID NO: 25 - DNA, 22 bp, synthetic construct
  source 1..22: mol_type = other DNA
gggtgactcattgagactgctc

SEQ ID NO: 26 - DNA, 22 bp, synthetic construct
  source 1..22: mol_type = other DNA
gcagatgactcgttcactggtt

SEQ ID NO: 27 - DNA, 22 bp, synthetic construct
  source 1..22: mol_type = other DNA
cgaagaacgatgaagagtgcgt

SEQ ID NO: 28 - DNA, 22 bp, synthetic construct
  source 1..22: mol_type = other DNA
gttcttaaaggactccggccat

SEQ ID NO: 29 - DNA, 22 bp, synthetic construct
  source 1..22: mol_type = other DNA
actgaaccacctctttacagcc

SEQ ID NO: 30 - DNA, 22 bp, synthetic construct
  source 1..22: mol_type = other DNA
tcttctcccattctttgtcgcg

SEQ ID NO: 31 - DNA, 21 bp, synthetic construct
  source 1..21: mol_type = other DNA
caaagcaaacagaggccaacg

SEQ ID NO: 32 - DNA, 21 bp, synthetic construct
  source 1..21: mol_type = other DNA
tttcgggggagatgatgcatc

SEQ ID NO: 33 - DNA, 22 bp, synthetic construct
  source 1..22: mol_type = other DNA
cagaccttcccaggagtctgta

SEQ ID NO: 34 - DNA, 22 bp, synthetic construct
  source 1..22: mol_type = other DNA
cagtatcttgccgtcaacgctt

SEQ ID NO: 35 - DNA, 22 bp, synthetic construct
  source 1..22: mol_type = other DNA
catggctagcgtccgattcttt

SEQ ID NO: 36 - DNA, 22 bp, synthetic construct
  source 1..22: mol_type = other DNA
ggtgcctgaagagacatagctg

SEQ ID NO: 37 - DNA, 22 bp, synthetic construct
  source 1..22: mol_type = other DNA
ctagacagggcaggtgggtata

SEQ ID NO: 38 - DNA, 20 bp, synthetic construct
  source 1..20: mol_type = other DNA
agcactgactggcgtactga

SEQ ID NO: 39 - DNA, 20 bp, synthetic construct
  source 1..20: mol_type = other DNA
atatccggcgcctgtgtact

SEQ ID NO: 40 - DNA, 22 bp, synthetic construct
  source 1..22: mol_type = other DNA
gccaagaactcattgcatgctg

SEQ ID NO: 41 - DNA, 22 bp, synthetic construct
  source 1..22: mol_type = other DNA
aactgcaacgtcacacagatga

SEQ ID NO: 42 - DNA, 23 bp, synthetic construct
  source 1..23: mol_type = other DNA
agtattcttggttgcatgcgaat

SEQ ID NO: 43 - DNA, 21 bp, synthetic construct
  source 1..21: mol_type = other DNA
tatacaggcggctgaaccctt

SEQ ID NO: 44 - DNA, 24 bp, synthetic construct
  source 1..24: mol_type = other DNA
gtatgtacattgggtaggaggacg

SEQ ID NO: 45 - DNA, 22 bp, synthetic construct
  source 1..22: mol_type = other DNA
aagttcggcgccatgatgaaat

SEQ ID NO: 46 - DNA, 22 bp, synthetic construct
  source 1..22: mol_type = other DNA
cggctggcgatggtgatattta

SEQ ID NO: 47 - DNA, 20 bp, synthetic construct
  source 1..20: mol_type = other DNA
gcagacccgctaaaaaggct

SEQ ID NO: 48 - DNA, 20 bp, synthetic construct
  source 1..20: mol_type = other DNA
agccagcgctcgtcttctat

SEQ ID NO: 49 - DNA, 22 bp, synthetic construct
  source 1..22: mol_type = other DNA
gcagaagccgacagtaagtacc

SEQ ID NO: 50 - DNA, 21 bp, synthetic construct
  source 1..21: mol_type = other DNA
aggctggtacctcctgttgta

SEQ ID NO: 51 - DNA, 24 bp, synthetic construct
  source 1..24: mol_type = other DNA
accagctcaaaagaaaaagaagcc

SEQ ID NO: 52 - DNA, 22 bp, synthetic construct
  source 1..22: mol_type = other DNA
accttcgtgcttgacttcgaaa

SEQ ID NO: 53 - DNA, 22 bp, synthetic construct
  source 1..22: mol_type = other DNA
cggagcagtacagtactcagga

SEQ ID NO: 54 - DNA, 22 bp, synthetic construct
  source 1..22: mol_type = other DNA
gcgtcccttgttgtcgaagatc

SEQ ID NO: 55 - DNA, 22 bp, synthetic construct
  source 1..22: mol_type = other DNA
aacgtcatgagacctgggtact

SEQ ID NO: 56 - DNA, 20 bp, synthetic construct
  source 1..20: mol_type = other DNA
aagttgtccttggtgctgcg

SEQ ID NO: 57 - DNA, 22 bp, synthetic construct
  source 1..22: mol_type = other DNA
ggccgggctatttgtaagaaca

SEQ ID NO: 58 - DNA, 22 bp, synthetic construct
  source 1..22: mol_type = other DNA
ccgtcagagtttctcctttcgg

SEQ ID NO: 59 - DNA, 22 bp, synthetic construct
  source 1..22: mol_type = other DNA
gatcgcacattgctgtcacaac

SEQ ID NO: 60 - DNA, 28 bp, synthetic construct
  source 1..28: mol_type = other DNA
tgtagttattagtccttcatttgagcca

SEQ ID NO: 61 - DNA, 22 bp, synthetic construct
  source 1..22: mol_type = other DNA
actgtatcctgaccacccaaca

SEQ ID NO: 62 - DNA, 22 bp, synthetic construct
  source 1..22: mol_type = other DNA
gcgtcacccactcttcttgata

SEQ ID NO: 63 - DNA, 21 bp, synthetic construct
  source 1..21: mol_type = other DNA
tatggcagtggggatgtgcat

SEQ ID NO: 64 - DNA, 22 bp, synthetic construct
  source 1..22: mol_type = other DNA
ttaggctaagcaggaaagggac

SEQ ID NO: 65 - DNA, 22 bp, synthetic construct
  source 1..22: mol_type = other DNA
acgtaacagtgatcccgaacac

SEQ ID NO: 66 - DNA, 22 bp, synthetic construct
  source 1..22: mol_type = other DNA
agctccatctccagtaccatgg

SEQ ID NO: 67 - DNA, 22 bp, synthetic construct
  source 1..22: mol_type = other DNA
gcacatgtggagaagtccgaat

SEQ ID NO: 68 - DNA, 22 bp, synthetic construct
  source 1..22: mol_type = other DNA
ttagctgatgcggatgcggtat

SEQ ID NO: 69 - DNA, 22 bp, synthetic construct
  source 1..22: mol_type = other DNA
gcctgagagcaaagacgtctat

SEQ ID NO: 70 - DNA, 22 bp, synthetic construct
  source 1..22: mol_type = other DNA
cctgagagtatggcacgtgtac

SEQ ID NO: 71 - DNA, 22 bp, synthetic construct
  source 1..22: mol_type = other DNA
tgcacccattcctcagactttg

SEQ ID NO: 72 - DNA, 22 bp, synthetic construct
  source 1..22: mol_type = other DNA
gatagtgacggcgttggtcatc

SEQ ID NO: 73 - DNA, 22 bp, synthetic construct
  source 1..22: mol_type = other DNA
tgtcatgggtgcagaagatcac

SEQ ID NO: 74 - DNA, 22 bp, synthetic construct
  source 1..22: mol_type = other DNA
cgtcaagttagtgcctgctgaa

SEQ ID NO: 75 - DNA, 30 bp, synthetic construct
  source 1..30: mol_type = other DNA
acccctgaatagtaacaaaatacaaaatca

SEQ ID NO: 76 - DNA, 26 bp, synthetic construct
  source 1..26: mol_type = other DNA
gccctttgaactacttctgtttatgg

SEQ ID NO: 77 - DNA, 22 bp, synthetic construct
  source 1..22: mol_type = other DNA
aacatctcctacgtccctgtgg

SEQ ID NO: 78 - DNA, 22 bp, synthetic construct
  source 1..22: mol_type = other DNA
ctttttgaaggccctgcaacgt

SEQ ID NO: 79 - DNA, 22 bp, synthetic construct
  source 1..22: mol_type = other DNA
acggccatcacctcttgtaagt

SEQ ID NO: 80 - DNA, 22 bp, synthetic construct
  source 1..22: mol_type = other DNA
tgtcggacaggaagtaccactg

SEQ ID NO: 81 - DNA, 22 bp, synthetic construct
  source 1..22: mol_type = other DNA
ctcatccgcccaatttgtcgag

SEQ ID NO: 82 - DNA, 22 bp, synthetic construct
  source 1..22: mol_type = other DNA
gtactgggtagggttcgacaca

SEQ ID NO: 83 - DNA, 22 bp, synthetic construct
  source 1..22: mol_type = other DNA
cacacgaaaccactgtgtcaca

SEQ ID NO: 84 - DNA, 22 bp, synthetic construct
  source 1..22: mol_type = other DNA
tcggtgttccatctaaagggca

SEQ ID NO: 85 - DNA, 22 bp, synthetic construct
  source 1..22: mol_type = other DNA
cttcatggtgttcgtgttccgt

SEQ ID NO: 86 - DNA, 22 bp, synthetic construct
  source 1..22: mol_type = other DNA
ggtggggctgaaccagagaata

SEQ ID NO: 87 - DNA, 22 bp, synthetic construct
  source 1..22: mol_type = other DNA
tacaaagctgtcgaattcggcc

SEQ ID NO: 88 - DNA, 22 bp, synthetic construct
  source 1..22: mol_type = other DNA
aagcagaaaacacacacggtct

SEQ ID NO: 89 - DNA, 22 bp, synthetic construct
  source 1..22: mol_type = other DNA
gcgcctctcggagtctctatta

SEQ ID NO: 90 - DNA, 22 bp, synthetic construct
  source 1..22: mol_type = other DNA
tacaggcagcacaggaagatgt

SEQ ID NO: 91 - DNA, 22 bp, synthetic construct
  source 1..22: mol_type = other DNA
tggaagtcttcgggcgaaattg

SEQ ID NO: 92 - DNA, 22 bp, synthetic construct
  source 1..22: mol_type = other DNA
ggagcaagtgaagacgtgtacg

SEQ ID NO: 93 - DNA, 22 bp, synthetic construct
  source 1..22: mol_type = other DNA
tatatggacaagcggggcgaat

SEQ ID NO: 94 - DNA, 22 bp, synthetic construct
  source 1..22: mol_type = other DNA
tgcaagaaggaagaagctgcag

SEQ ID NO: 95 - DNA, 22 bp, synthetic construct
  source 1..22: mol_type = other DNA
aaggcgatcaaggcaagtaacg

SEQ ID NO: 96 - DNA, 22 bp, synthetic construct
  source 1..22: mol_type = other DNA
atctgcacgtacggttgattcg

SEQ ID NO: 97 - DNA, 22 bp, synthetic construct
  source 1..22: mol_type = other DNA
ggttttgttgagcccgtagtgt

SEQ ID NO: 98 - DNA, 22 bp, synthetic construct
  source 1..22: mol_type = other DNA
ggcaaaatgcgcactacgaatg

SEQ ID NO: 99 - DNA, 22 bp, synthetic construct
  source 1..22: mol_type = other DNA
ccttccgtacgcgttaggagta

SEQ ID NO: 100 - DNA, 23 bp, synthetic construct
  source 1..23: mol_type = other DNA
aggagtttacgcagttaggcaaa

SEQ ID NO: 101 - DNA, 22 bp, synthetic construct
  source 1..22: mol_type = other DNA
ttcattcagggctacttcgggt

SEQ ID NO: 102 - DNA, 22 bp, synthetic construct
  source 1..22: mol_type = other DNA
tggtccctatcctcgaaacagc

SEQ ID NO: 103 - DNA, 22 bp, synthetic construct
  source 1..22: mol_type = other DNA
tcctggtagtcacgcagatctg

SEQ ID NO: 104 - DNA, 22 bp, synthetic construct
  source 1..22: mol_type = other DNA
tgaggcagcgtccattctagaa

SEQ ID NO: 105 - DNA, 22 bp, synthetic construct
  source 1..22: mol_type = other DNA
atacctaccaagtgttgccggt

SEQ ID NO: 106 - DNA, 23 bp, synthetic construct
  source 1..23: mol_type = other DNA
accttgcactgcctactaagaga

SEQ ID NO: 107 - DNA, 23 bp, synthetic construct
  source 1..23: mol_type = other DNA
acatggtggtttcaatgctctgg

SEQ ID NO: 108 - DNA, 22 bp, synthetic construct
  source 1..22: mol_type = other DNA
tcagagcatacggttacgcaga

SEQ ID NO: 109 - DNA, 22 bp, synthetic construct
  source 1..22: mol_type = other DNA
tttgcggttcctactggtgttg

SEQ ID NO: 110 - DNA, 22 bp, synthetic construct
  source 1..22: mol_type = other DNA
gacggtgtttgcaaggcagtat

SEQ ID NO: 111 - DNA, 22 bp, synthetic construct
  source 1..22: mol_type = other DNA
tccatggctgtaaagaggtggt

SEQ ID NO: 112 - DNA, 22 bp, synthetic construct
  source 1..22: mol_type = other DNA
acctctcctctccacaggtgta

SEQ ID NO: 113 - DNA, 22 bp, synthetic construct
  source 1..22: mol_type = other DNA
gcctctatttgctttggccaca

SEQ ID NO: 114 - DNA, 22 bp, synthetic construct
  source 1..22: mol_type = other DNA
aagggacacgttttcaccagac

SEQ ID NO: 115 - DNA, 24 bp, synthetic construct
  source 1..24: mol_type = other DNA
ggagcatttgactttttgcactcc

SEQ ID NO: 116 - DNA, 22 bp, synthetic construct
  source 1..22: mol_type = other DNA
cgacttcgcatgaaccatgtca

SEQ ID NO: 117 - DNA, 22 bp, synthetic construct
  source 1..22: mol_type = other DNA
gtatgtatcgccccgtcgtcta

SEQ ID NO: 118 - DNA, 22 bp, synthetic construct
  source 1..22: mol_type = other DNA
agtttgatctaagcgccgatgg

SEQ ID NO: 119 - DNA, 22 bp, synthetic construct
  source 1..22: mol_type = other DNA
aaatgtgatggggaacgtctcg

SEQ ID NO: 120 - DNA, 22 bp, synthetic construct
  source 1..22: mol_type = other DNA
cgcgtgacacagctatttcctt

SEQ ID NO: 121 - DNA, 22 bp, synthetic construct
  source 1..22: mol_type = other DNA
tggcgtaccgacttctgttgta

SEQ ID NO: 122 - DNA, 22 bp, synthetic construct
  source 1..22: mol_type = other DNA
acacggacgacgagttatgact

SEQ ID NO: 123 - DNA, 22 bp, synthetic construct
  source 1..22: mol_type = other DNA
gacaatcggacgttgatcggag

SEQ ID NO: 124 - DNA, 22 bp, synthetic construct
  source 1..22: mol_type = other DNA
taatggcagagaccccgaaagt

SEQ ID NO: 125 - DNA, 22 bp, synthetic construct
  source 1..22: mol_type = other DNA
tgtgttctggaatggggaaggt

SEQ ID NO: 126 - DNA, 22 bp, synthetic construct
  source 1..22: mol_type = other DNA
cgagcgacattcaatccgtcaa

SEQ ID NO: 127 - DNA, 22 bp, synthetic construct
  source 1..22: mol_type = other DNA
cggcagcagattatgggttctt

SEQ ID NO: 128 - DNA, 22 bp, synthetic construct
  source 1..22: mol_type = other DNA
gaagaatttgctgccagcccta

SEQ ID NO: 129 - DNA, 22 bp, synthetic construct
  source 1..22: mol_type = other DNA
gtgcggctataatggcatcgaa

SEQ ID NO: 130 - DNA, 22 bp, synthetic construct
  source 1..22: mol_type = other DNA
ttaggagattgaacgccgtcct

SEQ ID NO: 131 - DNA, 22 bp, synthetic construct
  source 1..22: mol_type = other DNA
gtcagacgatcttccaacaccc

SEQ ID NO: 132 - DNA, 22 bp, synthetic construct
  source 1..22: mol_type = other DNA
gacaggtacgcgcttcaagttc

SEQ ID NO: 133 - DNA, 22 bp, synthetic construct
  source 1..22: mol_type = other DNA
cgctaacggtttgcccagttta

SEQ ID NO: 134 - DNA, 22 bp, synthetic construct
  source 1..22: mol_type = other DNA
agctccctacttttgtggaggg

SEQ ID NO: 135 - DNA, 22 bp, synthetic construct
  source 1..22: mol_type = other DNA
gaccgccgtacaaggttatgac

SEQ ID NO: 136 - DNA, 22 bp, synthetic construct
  source 1..22: mol_type = other DNA
taatgtccatggccacctttgc

SEQ ID NO: 137 - DNA, 22 bp, synthetic construct
  source 1..22: mol_type = other DNA
tattcttccgattcttgcgcgg

SEQ ID NO: 138 - DNA, 22 bp, synthetic construct
  source 1..22: mol_type = other DNA
caacttgcccagctgatctcag

SEQ ID NO: 139 - DNA, 22 bp, synthetic construct
  source 1..22: mol_type = other DNA
atcgatggtcccctttacgtgt

SEQ ID NO: 140 - DNA, 22 bp, synthetic construct
  source 1..22: mol_type = other DNA
taaggtaacaggttacgcgtgc

SEQ ID NO: 141 - DNA, 22 bp, synthetic construct
  source 1..22: mol_type = other DNA
tacgggctccttcattagctcc

SEQ ID NO: 142 - DNA, 22 bp, synthetic construct
  source 1..22: mol_type = other DNA
gaccgatcttcgacaacaaggg

SEQ ID NO: 143 - DNA, 22 bp, synthetic construct
  source 1..22: mol_type = other DNA
gcgtcggggagaacatgttaag

SEQ ID NO: 144 - DNA, 22 bp, synthetic construct
  source 1..22: mol_type = other DNA
gagaaaaccttgcgcatgcttg

SEQ ID NO: 145 - DNA, 22 bp, synthetic construct
  source 1..22: mol_type = other DNA
ctgctggcatgtgattgtccat

SEQ ID NO: 146 - DNA, 22 bp, synthetic construct
  source 1..22: mol_type = other DNA
ccaggtttccttgcaaatcgga

SEQ ID NO: 147 - DNA, 22 bp, synthetic construct
  source 1..22: mol_type = other DNA
gtacgtgctgcaaggtagttcc

SEQ ID NO: 148 - DNA, 22 bp, synthetic construct
  source 1..22: mol_type = other DNA
catgtacgcacccatttcacca

SEQ ID NO: 149 - DNA, 22 bp, synthetic construct
  source 1..22: mol_type = other DNA
ggtccccgaattcagcattacg

SEQ ID NO: 150 - DNA, 22 bp, synthetic construct
  source 1..22: mol_type = other DNA
ctgcaaggtcgatcaatgccat

SEQ ID NO: 151 - DNA, 22 bp, synthetic construct
  source 1..22: mol_type = other DNA
tcattgttaccccacgtgacct

SEQ ID NO: 152 - DNA, 22 bp, synthetic construct
  source 1..22: mol_type = other DNA
agtgggtgacgcataagaagga

SEQ ID NO: 153 - DNA, 22 bp, synthetic construct
  source 1..22: mol_type = other DNA
tggtgtcagttcgtacggtgta

SEQ ID NO: 154 - DNA, 22 bp, synthetic construct
  source 1..22: mol_type = other DNA
atactcctgtcgatggtgggtg

SEQ ID NO: 155 - DNA, 22 bp, synthetic construct
  source 1..22: mol_type = other DNA
cgggatcactgttacgtgttcg

SEQ ID NO: 156 - DNA, 22 bp, synthetic construct
  source 1..22: mol_type = other DNA
tttttagccgtactgagcgtcg

SEQ ID NO: 157 - DNA, 22 bp, synthetic construct
  source 1..22: mol_type = other DNA
attttcggtgtcgcagaagcag

SEQ ID NO: 158 - DNA, 22 bp, synthetic construct
  source 1..22: mol_type = other DNA
gcggtacagcagagtgtaagga

SEQ ID NO: 159 - DNA, 22 bp, synthetic construct
  source 1..22: mol_type = other DNA
gtcgcctttgtacaccacgatt

SEQ ID NO: 160 - DNA, 22 bp, synthetic construct
  source 1..22: mol_type = other DNA
ccatgccgtcacagttaaggac

SEQ ID NO: 161 - DNA, 22 bp, synthetic construct
  source 1..22: mol_type = other DNA
agttcatcgctcttaccgggtt

SEQ ID NO: 162 - DNA, 22 bp, synthetic construct
  source 1..22: mol_type = other DNA
attggctaaaagaacgaggggc

SEQ ID NO: 163 - DNA, 22 bp, synthetic construct
  source 1..22: mol_type = other DNA
ttcccgaatagtgacggcgtta

SEQ ID NO: 164 - DNA, 22 bp, synthetic construct
  source 1..22: mol_type = other DNA
gcacccattcctcagactttgg

SEQ ID NO: 165 - DNA, 22 bp, synthetic construct
  source 1..22: mol_type = other DNA
cctgctaaacgacacgcatagc